(19) United States
(12) Patent Application Publication
(10) Pub. No.: US 2009/0304663 A1
(43) Pub. Date: Dec. 10, 2009

(54) USE OF GSK-3 INHIBITORS FOR THE TREATMENT OF PROSTATE CANCER

(75) Inventor: Robert Martin Kypta, London (GB)

Correspondence Address: MARSHALL, GERSTEIN & BORUN LLP, 233 SOUTH WACKER DRIVE, 6300 SEARS TOWER, CHICAGO, IL, 60606-6357, US

(73) Assignee: IMPERIAL INNOVATIONS LIMITED, London (GB)

(21) Appl. No.: 11/660,293

(22) PCT Filed: Aug. 17, 2005
(86) PCT No.: PCT/GB2005/003212

§ 371 Date: Feb. 27, 2008

(30) Foreign Application Priority Data
Aug. 17, 2004 (GB) 0418328.1

(51) Int. Cl. A61K 38/00; A61P 35/00; A61K 31/7088; A61K 38/45; A61K 48/00; A61K 31/167; A61K 38/17

(52) U.S. Cl. 424/94.5; 514/44 R; 514/2; 514/44 A; 514/625; 514/618; 514/629; 514/15

(57) ABSTRACT

A method of combating prostate cancer in a mammalian individual, the method comprising administering an inhibitor of glycogen synthase kinase-3 (GSK-3), or a polynucleotide which encodes an inhibitor of GSK-3, to the individual. A further anti-cancer agent can also be administered. Use of an inhibitor of GSK-3, or a polynucleotide which encodes an inhibitor of GSK-3, in the preparation of a medicament for combating prostate cancer. The medicament may contain a further anti-cancer agent.

### FIELD OF THE INVENTION

The invention relates to methods and medicaments for inhibiting prostate cancer cell growth and for combating prostate cancer. In particular the invention relates to inhibitors of glycogen synthase kinase-3 for use in these methods and medicaments.

### BACKGROUND OF THE INVENTION

Cancer of the prostate is a very serious disease, second only to lung cancer in its level of mortality. Prostate cell growth and development are mediated by androgens and the androgen receptor (AR), a member of the nuclear receptor superfamily. Although patients with advanced prostate cancer are effectively treated with anti-androgen therapy (androgen ablation), the effect on disease, progression is usually only temporary, and ultimately prostate cancer can become unresponsive to androgen ablation. It is then classified as hormone-refractory (androgen independent) prostate cancer, which has no known cure. Therefore, the development of novel therapeutic agents is an urgent issue for prostate cancer treatment.

The transcriptional activity of AR is regulated by interaction with various co-regulators (reviewed by Cheshire & Isaacs, 2003; Cronauer et al, 2003), one of which is β-catenin. Interest in the role of β-catenin in prostate cancer has been stimulated by reports showing that it is aberrantly expressed in the cytoplasm and/or nucleus in up to 38% of hormone-refractory tumours. Evidence that increased levels of β-catenin lead to activation of AR transcriptional activity come largely from studies in which β-catenin is overexpressed (Chesire et al, 2002; Mulholland et al, 2002; Truica et al, 2000; Yang et al, 2002). However, the level of endogenous β-catenin is already very high in prostate cancer cells and stable expression of mutant β-catenin does not alter their proliferative response to androgen (Chesire et al, 2002). Therefore, it was believed to be important to determine how endogenous β-catenin affects AR transcriptional activity in prostate cancer cells.

Here we describe experiments in which we examined the effect of depleting endogenous β-catenin on androgen receptor activity using Axin and RNA interference. Axin, which promotes β-catenin degradation, inhibited androgen receptor transcriptional activity. However, this did not require the β-catenin binding domain of Axin. Depletion of β-catenin using RNA interference increased, rather than decreased, androgen receptor activity, suggesting that endogenous β-catenin is not a transcriptional coactivator for the androgen receptor.

Surprisingly and unexpectedly, our results show that glycogen synthase kinase-3 (GSK-3), rather than β-catenin, is an important endogenous regulator of AR transcriptional activity.

GSK-3 is a serine/threonine kinase known for its roles in glycogen metabolism and diabetes, in the Wnt signaling pathway, in the immune system, and in neurological disorders (reviewed by Doble & Woodgett (2003); Frame & Cohen (2001); Grimes & Jope (2001); and Woodgett (2001)). GSK-3 has been shown to be active in most resting cells and is subject to negative regulation by external stimuli. In response to growth factor stimulation, for example, kinases such as Akt inhibit GSK-3 by phosphorylation on serine 9 (Cross et al, 1995; Stambolic & Woodgett, 1994). In some instances, GSK-3 has been shown to be activated by agents that promote phosphorylation on tyrosine 216 (Bhat et al, 2000). GSK-3 can also be regulated by binding to the proteins Axin, FRAT (Frequently rearranged in advanced T-cell lymphomas)/GBP and the Kaposi's sarcoma-associated herpesvirus latency-associated nuclear antigen (Fujimuro et al, 2003; Ikeda et al, 1998; Yost et al, 1998). GSK-3 has numerous substrates, including a number of transcription factors such as c-Jun, c-myc, C/EBPs (CCAAT enhancer binding proteins) and NF-ATc (nuclear factor of activated T cells). The effects of phosphorylation by GSK-3 tend to be inhibitory and include promotion of degradation and enhancement of nuclear export (for references see Frame & Cohen (2001)). Thus, inhibition of GSK-3 often results in increased gene expression. However, there are examples where GSK-3 positively regulates gene expression, such as through CREB phosphorylation (Salas et al, 2003).

We now show that GSK-3 positively regulates AR transcriptional activity. The GSK-3-interaction domain of Axin prevents formation of a GSK-3-androgen receptor complex and is both necessary and sufficient for inhibition of androgen receptor dependent transcription. A second GSK-3-binding protein, FRAT, also inhibits androgen receptor transcriptional activity, as do the GSK-3 inhibitors SB216763 and SB415286. Finally, inhibition of GSK-3 reduces the growth of androgen receptor expressing prostate cancer cell lines. Since GSK-3 inhibitors inhibit the proliferation of prostate cancer cells, these drugs are expected to be1 useful in the treatment of patients with prostate cancer.

### SUMMARY OF THE INVENTION

A first aspect of the invention provides a method of combating prostate cancer in a mammalian individual, the method comprising administering an inhibitor of glycogen synthase kinase-3 (GSK-3), or a polynucleotide which encodes air inhibitor of GSK-3, to the individual.

In an embodiment, the inhibitor of GSK-3 is the only anti-cancer agent administered.

In an embodiment, the invention includes combating prostate cancer by administering an inhibitor of GSK-3, or a polynucleotide which encodes an inhibitor of GSK-3, to an individual who is not administered TRAIL. In other word, the invention does not include administering both an inhibitor of GSK-3 and TRAIL to an individual.

It is appreciated that the enzyme GSK-3 (EC 2.7.1.37) has two isoforms, GSK-3 α and GSK-3 β. Except where the context demands otherwise, by GSK-3 we include both GSK-3 α and GSK-3β.

By GSK-3 we include the meaning of a product of a human GSK-3 gene, including naturally occurring variants thereof. The cDNA sequence corresponding to a human GSK-3 β mRNA is found in Genbank Accession No. NM_{—}002093. Human GSK-3 β includes the amino acid sequence listed in Genbank Accession Nos. NM_{—}002093 and NP_{—}002084, and naturally occurring variants thereof. The cDNA sequence corresponding to a human GSK-3 α mRNA is found in Genbank Accession No. NM_{—}019884. Human GSK-3 α includes the amino acid sequence listed in Genbank Accession Nos. NNd_{—}019884 and NP_{—}063937, and naturally occurring variants thereof.

By GSK-3 we also include a homologous gene product from GSK-3 genes from other species.

It is preferred if the inhibitor of GSK-3 is selective for GSK-3 .

By a “selective” inhibitor of GSK-3 we include the meaning that the inhibitor has an IC₅₀ value for GSK-3 which is lower than for other protein kinases. Preferably, the GSK-3 selective inhibitor has an IC₅₀ value at least five or ten times lower than for at least one other protein kinase, and preferably more than 100 or 500 times lower. More preferably, the GSK-3 selective inhibitor has an IC₅₀ value more than 1000 or 5000 times lower than for at least one other protein kinase. Preferably, the at least one other protein kinase is a mammalian, more preferably human, protein kinase. Also preferably, the selective inhibitor of GSK-3 has a lower IC₅₀ value than for at least 2 or 3 or 4 or 5 or at least 10 other protein kinases. Methods for determining the selectivity of a GSK-3 inhibitor are described by Ring et al (2003) with respect to 20 different protein kinases, and the at least one other protein kinase may be any one or more of them.

It is preferred if a selective inhibitor of GSK-3 has an IC₅₀ value at least ten times lower than for cdc2, one of the most closely related kinases, and preferably at least 100, or 500 times lower. More preferably, the GSK-3 selective inhibitor has an IC₅₀ value more than 1000 or 5000 times lower for GSK-3 than for cdc2.

Most preferably, the GSK-3 selective inhibitor has an IC₅₀ value at least five, times lower than for all other human protein kinases, and preferably at least 10, 50, 100 or 500 times lower.

The inhibitor of GSK-3 may be a peptide or a non-peptide drug. The inhibitor may inhibit GSK-3 α or GSK-3 β or both. The inhibitor may be SB415286 from GlaxoSmithKline, or a related GSK-3 inhibitory compounds such as a 3-indolyl-4-phenyl-1H-pyrrole-2,5-dione derivative, or other pyridine or pyrimidine derivative from other companies.

Although lithium chloride is an inhibitor of GSK-3, it is preferred if the inhibitor of GSK-3 is not lithium chloride. Lithium chloride has a high IC₅₀ value and is known to inhibit inositol monophosphatases to a similar extent as it inhibits GSK-3.

Further examples of GSK-3 inhibitors are known to those skilled in the art. Examples are described in, for example, WO 99/65897 and WO 03/074072 and references cited therein. For example, various GSK-3 inhibitory compounds and methods of their synthesis and use are disclosed in U.S. and international patent application Publication Nos. US 20050054663, US 20020156087, WO 02/20495 and WO 99/65897 (pyrimidine and pyridine based compounds); US 20030008866, US 20010044436 and WO01/44246 (bicyclic based compounds); US 20010034051 (pyrazine based compounds); and WO 98/36528 (purine based compounds). Further GSK-3 inhibitory compounds include those disclosed in WO 02/22598 (quinolinone based compounds), US 20040077707 (pyrrole based compounds); US 20040138273 (carbocyclic compounds); US 20050004152 (thiazole compounds); and US 20040034037 (heteroaryl compounds).

Further GSK-3 inhibitory compounds include macrocyclic maleimide selective GSK-3 β inhibitors developed by Johnson & Johnson and described in, for example, Kuo et al (2003) J Med Chem 46(19): 4021-31. The bis-7-azamdolylmaleimides #28 and #29 are reported as exhibiting little or no inhibitions to a panel of 50 protein kinases. Compound #29 almost behaved as a GSK-3 β specific inhibitor. Both #28 and #29 displayed good potency in GS cell-based assay. A particular example is 10,11,13,14,16,17,19,20,22,23-Decahydro-9,4:24,29-dimetho-1H-dipyrido (2,3-n:3′,2′-t) pyrrolo (3,4-q)-(1,4,7,10,13,22) tetraoxadiazacyclotetracosine-1,3 (2H)-dione.

The Pharmaprojects database indicates further GSK-3 inhibitors being developed by the following companies: Cyclacel (UK), Xcellsyz (UK)—XD-4241, Vertex Pharmaceuticals (USA)—eg VX-608, Chiron (USA), eg CHIR-73911, Kinetek (Canada) eg KP-354.

Ring et al (2003) describes substituted aminopyrimidine derivatives CHIR 98014 and CHIR 99021 that inhibit human GSK-3 potently (Kᵢ 0.87 and 9.8 nmol/l, respectively) with at least 500-fold selectivity against 20 other protein kinases. Cline et al (2003) also describes a substituted aminopyrimidine derivative, CPIR 98023, that selectively inhibits human GSK-3 with a Kᵢ of 5 nmol/l.

Liao et al (2004) describes the use of GSK-3 inhibitors RO318220 and GF10923 X (see references 28 and 29 therein).

Pierce et al (2005) describes a quinazolin-4-ylthiazol-2-ylamine GSK-3 inhibitor that was designed to allow specific hydrogen bonding with the protein.

A number of other GSK-3 inhibitors which may be useful in the present invention are commercially available from Calbiochem®. For example:
AR-A014418 (N-(4-Methoxybenzyl)-N′-(5-nitro-1,3-thiazol-2-yl)urea (Catalogue No. 361549) is a cell-permeable thiazole-containing urea compound that acts as a potent, ATP-competitive, and highly specific inhibitor of GSK-3 β (IC₅₀=104 nM; Kᵢ=38 nM) whose specificity has been confirmed using a panel of 28 kinases, including Cdk2 and Cdk5 (IC₅₀>100 mM)(Bhat et al 2003).
5-Methyl-1H-pyrazol-3-yl)-(2-phenylquinazolin-4-yl)amine (Catalogue No. 361555) is an aminopyrazole compound that acts as a potent ATP-binding site inhibitor of GSK-3 with a Kᵢ of 24 nM (Pierce et al 2005).
TDZD-8 (4-Benzyl-2-methyl-1,2,4-thiadiazolidine-3,5-dione; Catalogue No. 361540) is a highly selective, non-ATP competitive inhibitor of GSK-3 β (IC₅₀=2 mM) that does not significantly affect the activities of Cdk-1/cyclin B, CK-II, PKA, and PKC (IC₅₀>100 mM) (Barry et al 2003; Martinez et al 2002).
2-Thio(3-iodobenzyl)-5-(1-pyridyl)-[1,3,4]-oxadiazole (Catalogue No. 361541) is a 2-thio-[1,3,4]-oxadiazole-pyridyl derivative that acts as a potent inhibitor of glycogen synthase kinase-3 β (IC₅₀=390 nM) (Naerum et al, 2002).
3-(1-(3-Hydroxypropyl)-1H-pyrrolo[2,3-b]pyridin-3-yl]-4-pyrazin-2-yl-pyrrole-2,5-dione (Catalogue No. 361553) is a cell-permeable azaindolylmaleimide compound that acts as a potent, specific, and ATP-competitive inhibitor of GSK-3 β (Kᵢ=25 nM) and minimally inhibits a panel of 79 commonly studied protein kinases, including several PKC isozymes (O'Neill et al 2004).
(2′Z,3′E)-6-Bromoindirubin-3′-oxime (Catalogue No. 361550) is a cell-permeable bis-indolo (indirubin) compound that acts as a highfy potent, selective, reversible, and ATP-competitive inhibitor of GSK-3 α/β (IC₅₀=5 nM) whose specificity has been tested against various Cdk's (IC₅₀=83, 300, 320, and 10,000 nM for Cdk5/p25, Cdk2/A, Cdk1/B, and Cdk4/D1, respectively) as well as many other commonly studied kinases (IC₅₀≧10 μM), including MAP kinases, PKA, PKC isoforms, PKG, CK, and IRTK (Polychronopoulos et al 2004; Sato et al 2004; Meijer et al 2003).
BIO-Acetoxime ((2′Z,3′E)-6-Bromoindirubin-3′-acetoxime; Catalogue No. 361551) exhibits greater selectivity for GSK-3 α/β (IC₅₀=0.01 mM) than for Cdk5/p25. Cdk2/A and Cdk1/B (IC₅₀=2.4 mM, 4.3 mM and 63 mM, respectively). It weakly affects the activities of Cdk4/D1 and many other kinases (IC₅₀≧10 mM) (Knockaert et al 2004; Polychronopoulos et al 2004; Meijer et al 2003).
L803-mts, supplied as a trifluoroacetate salt, is a cell-permeable yristoylated peptide GSK-3 β inhibitor (Catalogue No. 361546) which acts as a selective, substrate-specific, competitive inhibitor of GSK-3 β (IC₅₀=40 mM) and which displays in vivo stability. It does not affect the activities of Cdc2, PKB, and PKC (Plotkin et al 2003).
1-Azakenpaullone (Catalogue No. 191500) acts as a potent and ATP-competitive inhibitor of GSK-3 b (IC₅₀=18 nM), and displays ˜100-200 fold greater selectivity over Cdk1/B and Cdk5/p25 (IC₅₀=2.0 mM and 4.2 mM, respectively) (Kunick et al 2004).

The GSK-3 inhibitor may be a small interfering RNA (siRNA; Harmon et al. Nature, 418 (6894): 244-51 (2002); Brummelkamp et al, Science 21, 21 (2002); and Sui et al, Proc. Natl. Acad. Sci. USA 99, 5515-5520 (2002)). RNA interference (RNAi) is the process of sequence-specific post-transcriptional gene silencing in animals initiated by double-stranded (dsRNA) that is homologous in sequence to the silenced gene. The mediators of sequence-specific mRNA degradation are typically 21- and 22-nucleotide small interfering RNAs (siRNAs) which, in vivo, may be generated by ribonuclease in cleavage from longer dsRNAs. 21-nucleotide siRNA duplexes have been shown to specifically suppress expression of both endogenous and heterologous genes (Elbashir et al (2001) Nature 411:494-498). In mammalian cells it is believed that the siRNA has to be comprised of two complementary 21 mers as described below since longer double-stranded (ds) RNAs will activate PKR (dsRNA-dependent protein kinase) and inhibit overall protein synthesis.

Duplex siRNA molecules selective for GSK3 α and GSK3 β can readily be designed by reference to their cDNA sequence. Typically, the first 21-mer sequence that begins with an AA dinucleotide which is at least 120 nucleotides downstream from the initiator methionine codon is selected. The RNA sequence perfectly complementary to this becomes the first RNA oligonucleotide. The second RNA sequence should be perfectly complementary to the first 19 residues of the first, with an additional UU dinucleotide at its 3′ end. Once designed, the synthetic RNA molecules can be synthesised using methods well known in the art.

Liao et al (2003) describe siRNA molecules that act as specific GSK-3 β inhibitors. These include (5′-3′) AAG AAT CGA GAG CTC CAG ATC (SEQ ID NO: 1) and AAG TAA TCC ACC TCT GGC TAG (SEQ ID NO: 2). GSK-3 siRNAs have been described by a number of other groups including Yu et al. (2003; Mol Ther. 7(2): 228-36) and there are commercially available GSK-3 (α and β) siRNAs (Upstate). Additional GSK-3 specific siRNA molecules can readily be identified and prepared by a person of skill in the art.

Other specific GSK-3 inhibitors include antisense or triplet-forming nucleic acid molecules or ribozymes. Antisense nucleic acid molecules and ribozymes selective for GSK-3 can be designed by reference to the cDNA or gene sequence, as is known in the art.

Further potential GSK-3 inhibitors include neutralising anti-GSK-2 antibodies, ie, those which inhibit the relevant biological activity of GSK-3. The term “antibody” includes but is not limited to polyclonal, monoclonal, chimeric, single chain, Fab fragments and fragments produced by a Fab expression library. Such fragments include fragments of whole antibodies which retain their binding activity for a target substance, Fv, F(ab′) and F(ab′)2 fragments, as well as single chain antibodies (scFv), fusion proteins and other synthetic proteins which comprise the antigen-binding site of the antibody. Furthermore, the antibodies and fragments thereof may be humanised antibodies, which are now well known in the art.

The documents indicated above relating to GSK-3 inhibitors are hereby specifically incorporated by reference.

Further GSK-3 inhibitors include the GSK-3-binding domain of Axin (also known as the GSK-3 interaction domain, GID) or a variant thereof that inhibits GSK-3, and the GSK-3-binding domain of FRAT or a variant thereof that inhibits GSK-3. Methods and assays for determining the rate or level of GSK-3 inhibition, and hence for determining whether and to what extent a compound inhibits GSK-3, are described in the Examples and in the documents listed above, for example Liao et al 2003, 2004).

By a “variant” of GID, and of the GSK-3-binding domain of FRAT, we include a fragment, sequence variant, modification or fusion, or combinations thereof, of either of these molecules.

It is appreciated that, technically, the GSK-3 binding domains of Axin and FRAT are not GSK-3 inhibitors since they have not been shown to inhibit the catalytic activity of GSK-3 kinase. However, they are believed to sequester GSK-3 preventing its interaction with AR, and in this way inhibit the activity of GSK-3.

The variants may be made using protein chemistry techniques for example using partial proteolysis (either exolytically or endolytically), or by de novo synthesis. Alternatively, the variants may be made by recombinant DNA technology. Suitable techniques for cloning, manipulation, modification and expression of nucleic acids, and purification of expressed proteins, are well known in the art and are described for example in Sambrook et al (2001) “Molecular Cloning, a Laboratory Manual”, 3^{rd} edition, Sambrook et al (eds), Cold Spring Harbor Laboratory Press, Cold Spring Harbor, N.Y., USA, incorporated herein by reference.

Of the prostate cancer cell lines tested, the ones that do not respond to GSK-3 inhibitors are PC3 and DU145, neither of which express AR. Without being bound by theory, we appreciate that GSK-3 inhibitors may not work in patients with tumours that do not express the AR. Thus, typically and preferably, the individual is one with prostate cancer that expresses AR.

In an embodiment, the invention includes the prior step of determining if the prostate cancer expresses AR. However, since prostate cancer that does not expresses AR is rare (fewer than 10% of cases), this prior step may not be necessary.

We have also shown that GSK-3 inhibitors inhibit the growth of androgen-dependent (AD) prostate cancer cell lines such as LNCaP, as well as prostate cancer cell lines such as 22Rv1 that are androgen-independent (AI).

Thus a GSK-3 inhibitor may be useful in combating AD prostate cancer. A GSK-3 inhibitor may also be useful in combating AI prostate cancer.

In an embodiment, the invention includes the prior step of determining if the prostate cancer is AD or AI. Since GSK-3 inhibitors can be used to combat both AD and AI prostate cancer, this prior step may not be necessary. However, AD cancer is susceptible to anti-androgen therapy; this therapy is effective in the majority of patients for about 2 years before the tumour becomes AI, when such therapy no longer works. Therefore, determining the androgen sensitivity status of the prostate cancer may be important in determining an appropriate additional therapeutic agent to be administered. Methods for determining whether prostate cancer is AD or AI are well known to a person of skill in the art.

As shown in the examples, a GSK-3 inhibitor has a reduced effect on LNCaP cells, and we believe that this is because GSK-3 is not very active in this cell line. Without being bound by theory, we expect that the GSK-3 inhibitor will be therapeutically useful in patients who's prostate cancer has active GSK-3. The activity of GSK-3 can be measured directly by kinase assay, or indirectly by staining with commercially available antibodies (antibodies to GSK-3 phosphorylated on serine 9 recognise less active GSK-3; antibodies to GSK-3 phosphorylated on tyrosine 216 recognise more active GSK-3) as is well known to a person of skill in the art.

Preferably, the mammalian individual is a human. Alternatively, the individual may be an animal, for example a domesticated animal (for example a dog or cat), laboratory animal (for example laboratory rodent, for example mouse, rat or rabbit) or animal important in agriculture (ie livestock), for example cattle, sheep or goats.

By “combating” prostate cancer we include the meaning that the invention can be used to alleviate symptoms of the disorder (ie palliative use), or to treat the disorder, or to prevent the disorder (ie prophylactic use).

The inhibitor of GSK-3, or a formulation thereof, may be administered by any conventional method including oral and parenteral (eg subcutaneous or intramuscular) injection. Preferred routes include oral, intranasal or intramuscular injection. Routes already known for GSK-3 inhibitors may be used, though it will be appreciated that different localised treatment routes may be more appropriate in combating prostate cancer than for when treating (for example) diabetes. The treatment may consist of a single dose or a plurality of doses over a period of time.

In an embodiment, the inhibitor of GSK-3 may be targeted to the prostate non-specifically via the androgen receptor.

The GSK-3 inhibitor may be given to a subject who is being treated for the prostate cancer by some other method. Thus, although the method of treatment may be used alone it is desirable to use it as an adjuvant therapy, for example alongside conventional preventative, therapeutic or palliative methods. Such methods may include surgery, radiation therapy including brachytherapy, and chemotherapy.

Thus, in an embodiment, the GSK-3 inhibitor is administered to a patient who is also administered a further anti-cancer agent. Cancer chemotherapeutic agents include: alkylating agents including nitrogen mustards such as mechlorethamine (HN₂), cyclophosphamide, ifosfamide, melphalan (L-sarcolysin) and chlorambucil; ethylenimines and methylmelamines such as hexamethylmelamine, thiotepa; alkyl sulphonates such as busulfan: nitrosoureas such as carmustine (BCNU), lomustine (CCNU), semustine (methyl-CCNU) and streptozocin (streptozotocin); and triazenes such as decarbazine (DTIC; dimethyltriazenoimidazole-carboxamide); Antimetabolites including folic acid analogues such as methotrexate (amethopterin); pyrimidine analogues such as fluorouracil (5-fluorouracil; 5-FU), floxuridine (fluorodeoxyuridine; FUdR) and cytarabine (cytosine arabinoside); and purine analogues and related inhibitors such as mercaptopurine (6-mercaptopurine; 6-MP), thioguanine (6-thioguanine; TG) and pentostatin (2′-deoxycoformycin). Natural Products including vinca alkaloids such as vinblastine (VLB) and vincristine; epipodophyllotoxins such as etoposide and teniposide; antibiotics such as dactinomycin (actinomycin D), daunorubicin (daunomycin; rubidomycin), doxorubicin, bleomycin, plicamycin (mithramycin) and mitomycin (mitomycin C); enzymes such as L-asparaginase; and biological response modifiers such as interferon alphenomes. Miscellaneous agents including platinum coordination complexes such as cisplatin (cis-DDP) and carboplatin; anthracenedione such as mitoxantrone and anthracycline; substituted urea such as hydroxyurea; methyl hydrazine derivative such as procarbazine (N-methylhydrazine, MTH); and adrenocortical suppressant such as mitotane (o,p′-DDD) and aminoglutethimide; taxol and analogues/derivatives; and hormone agonists/antagonists such as flutamide and tamoxifen.

Other suitable agents include GnRH and analogues thereof; both GnRH agonists and antagonists can act to lower serum androgen levels.

It is preferred, however, if the further anti-cancer agent is selected from GnRH agonists such as leuprorelin, goserelin, and buserelin, anti-androgens such as bicalutamide and flutamide, steroids such as hydrocortisone, prednisone and dexamethasone, and chemotherapy agents such as mitozantrone, estramustine and docetaxol (Schellhammer & Davis 2004; Assikis & Simons, 2004; Gulley & Daliut, 2004).

Typically, if the prostate cancer is AD, the further anti-cancer agent is an anti-androgen.

In an embodiment, the further an anti-cancer agent is not TRAIL. In other words, the invention includes combating prostate cancer in an individual by administering a GSK-3 inhibitor and a further anti-cancer agent other than TRAIL.

Whilst it is possible for a therapeutic molecule as described herein, to be administered alone, it is preferable to present it as a pharmaceutical formulation, together with one or more acceptable carriers. The carrier(s) must be “acceptable” in the sense of being compatible with the therapeutic molecule and not deleterious to the recipients thereof. Typically, the carriers will be water or saline which will be sterile and pyrogen free.

The formulations may conveniently be presented in unit dosage form and may be prepared by any of the methods well known in the art of pharmacy. Such methods include the step of bringing into association the active ingredient (for an antigenic molecule, construct or chimeric polypeptide of the invention) with the carrier which constitutes one or more accessory ingredients. In general the formulations are prepared by uniformly and intimately bringing into association the active ingredient with liquid carriers or finely divided solid carriers or both, and then, if necessary, shaping the product.

Formulations in accordance with the present invention suitable for oral administration may be presented as discrete units such as capsules, cachets or tablets, each containing a predetermined amount of the active ingredient; as a powder or granules; as a solution or a suspension in an aqueous liquid or a non-aqueous liquid; or as an oil-in-water liquid emulsion or a water-in-oil liquid emulsion. The active ingredient may also be presented as a bolus, electuary or paste.

A tablet may be made by compression or molding, optionally with one or more accessory ingredients. Compressed tablets may be prepared by compressing in a suitable machine the active ingredient in a free-flowing form such as a powder or granules, optionally mixed with a binder (eg povidone, gelatin, hydroxypropylmethyl cellulose), lubricant, inert diluent, preservative, disintegrant (eg sodium starch glycolate, cross-linked povidone, cross-linked sodium carboxymethyl cellulose), surface-active or dispersing agent. Molded tablets may be made by moulding in a suitable machine a mixture of the powdered compound moistened with an inert liquid diluent. The tablets may optionally be coated or scored and may be formulated so as to provide slow or controlled release of the active ingredient therein using, for example, hydroxypropylmethylcellulose in varying proportions to provide desired release profile.

Formulations suitable for topical administration in the mouth include lozenges comprising the active ingredient in a flavoured base, usually sucrose and acacia or tragacanth; pastilles comprising the active ingredient in an inert base such as gelatin and glycerin, or sucrose and acacia; and mouth-washes comprising the active ingredient in a suitable liquid carrier.

Formulations suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example sealed ampoules and vials, and may be stored in a freeze-dried (lyophilised) condition requiring only the addition of the sterile liquid carrier, for example water for injections, immediately prior to use. Extemporaneous injection solutions and suspensions may be prepared from sterile powders, granules and tablets of the kind previously described.

Preferred unit dosage formulations are those containing a daily dose or unit, daily sub-dose or an appropriate fraction thereof, of the GSK-3 inhibitor.

For example, the Ki for SB216763 is 3 μM and this was found to be the optimal dose for inhibition of prostate cancer growth in CWR-R1 cells. Lower doses of CHIR 98014 were administered to rats (Ring et al, 2003). The dose of the GSK-3 inhibitor to be administered is one mat provides an effective concentration at the prostate cancer of between 0.1 and 10 μM, preferably between 1 and 10 μM.

It should be understood that in addition to the ingredients particularly mentioned above, the formulations of this invention may include other agents conventional in the art having regard to the type of formulation in question, for example those suitable for oral administration may include flavouring agents.

In a preferred embodiment, the therapeutic molecules are administered orally.

It will be appreciated that the therapeutic molecule can be delivered to the area of the prostate by any means appropriate for localised administration of a drug. For example, a solution of the therapeutic molecule can be injected directly to the prostate or can be delivered by infusion using an infusion pump. The therapeutic molecule also can be incorporated into an implantable device which when placed at the desired site, permits the therapeutic molecule to be released into the surrounding locus.

The therapeutic molecule may be administered via a hydrogel material. The hydrogel is non-inflammatory and biodegradable. Many such materials now are known, including those made from natural and synthetic polymers. In a preferred embodiment, the method exploits a hydrogel which is liquid below body temperature but gels to form a shape-retaining semisolid hydrogel at or near body temperature. Preferred hydrogel are polymers of ethylene oxide-propylene oxide repeating units. The properties of the polymer are dependent on the molecular weight of the polymer and the relative percentage of polyethylene oxide and polypropylene oxide in the polymer. Preferred hydrogels contain from about 10% to about 80% by weight ethylene oxide and from about 20% to about 90% by weight propylene oxide. A particularly preferred hydrogel contains about 70% polyethylene oxide and 30% polypropylene oxide. Hydrogels which can be used are available, for example, from BASF Corp., Parsippany, N.J., under the tradename Pluronic®.

At present, there are no known surface antigens that are specific to the prostate. Prostate specific membrane antigen has a high degree of cross-reactivity with other epithelial cells in other organs. However, once a suitable prostate specific antigen has been identified, the inhibitor of GSK-3 may be targeted to the required site using a targeting moiety which binds to or lodges at the site of the prostate cancer. For example, the prostate could be targeted using a prostate-specific antibody with a cleavable linker to a GSK-3 inhibitor. A combined targeting/pro drug approach may be useful.

A pro-drug approach may also be used without targeting. Accordingly, reference to a GSK-3 inhibitor includes reference to a GSK-3 inhibitor prodrug.

It is appreciated that the GSK-3 inhibitor may itself be a polynucleotide, or may be encoded by a polynucleotide. Polynucleotides may be administered by any effective method, for example, parenterally (eg intravenously, subcutaneously, intramuscularly) or by oral, nasal or other means which permit the oligonucleotides to access and circulate in the patient's bloodstream. Polynucleotides administered systemically preferably are given in addition to locally administered polynucleotides, but also have utility in the absence of local administration. A dosage in the range of from about 0.1 to about 10 grams per administration to an adult human generally will be effective for this purpose.

The polynucleotide may be administered as a suitable genetic construct as is described below and delivered to the patient where it is expressed. Typically, the polynucleotide in the genetic construct is operatively linked to a promoter which can express the compound in the cell. The genetic constructs of the invention can be prepared using methods well known in the art, for example in Sambrook et al (2001).

Dendritic cell vaccine approaches may be useful in gene therapy for combating prostate cancer.

Although genetic constructs for delivery of polynucleotides can be DNA or RNA it is preferred if it is DNA.

Preferably, the genetic construct is adapted for delivery to a human cell.

Means and methods of introducing a genetic construct into a cell in an animal body are known in the art. For example, the constructs of the invention may be introduced into cells by any convenient method, for example methods involving retroviruses, so that the construct is inserted into the genome of the cell. For example, in Kuriyama et al (1991) Cell Struc. and Func. 16, 503-510 purified retroviruses are administered. Retroviral DNA constructs comprising a polynucleotide as described above may be made using methods well known in the art. To produce active retrovirus from such a construct it is usual to use an ecotropic psi2 packaging cell line grown in Dulbecco's modified Eagle's medium (DMEM) containing 10% foetal calf serum (FCS). Transfection of the cell line is conveniently by calcium phosphate co-precipitation, and stable transformants are selected by addition of G418 to a final concentration of 1 mg/ml (assuming the retroviral construct contains a neo^{R} gene). Independent colonies are isolated and expanded and the culture supernatant removed, filtered through a 0.45 μm pore-size filter and stored at −70° C. For the introduction of the retrovirus into the tumour cells, it is convenient to inject directly retroviral supernatant to which 10 μg/ml Polybrene has been added. For tumours exceeding 10 mm in diameter it is appropriate to inject between 0.1 ml and 1 ml of retroviral supernatant; preferably 0.5 ml.

Alternatively, as described in Culver et al (1992) Science 256, 1550-1552, cells which produce retroviruses are injected. The retrovirus-producing cells so introduced are engineered to actively produce retroviral vector particles so that continuous productions of the vector occurred within the tumour mass in situ. Thus, proliferating epidermal cells can be successfully transduced in vivo if mixed with retroviral vector-producing cells.

Targeted retroviruses are also available for use in the invention; for example, sequences conferring specific binding affinities may be engineered into pre-existing viral env genes (see Miller & Vile (1995) Faseb J. 9, 190-199 for a review of this and other targeted vectors for gene therapy).

Other methods involve simple delivery of the construct into the cell for expression therein either for a limited time or, following integration into the genome, for a longer time. An example of the latter approach includes liposomes (Nässander et al (1992) Cancer Res. 52, 646-653).

Other methods of delivery include adenoviruses carrying external DNA via an antibody-polylysine bridge (see Curiel (1993) Prog. Med. Virol. 40, 1-18) and transferrin-polycation conjugates as carriers (Wagner et al (1990) Proc. Natl. Acad. Sci. USA 87, 3410-3414). In the first of these methods a polycation-antibody complex is formed with the DNA construct or other genetic construct of the invention, wherein the antibody is specific for either wild-type adenovirus of a variant adenovirus in which a new epitope has been introduced which binds the antibody. The polycation moiety binds the DNA via electrostatic interactions with the phosphate backbone. The adenovirus, because it contains unaltered fibre and penton proteins, is internalised into the cell and carries into the cell with it the DNA construct of the invention. It is preferred if the polycation is poly lysine.

The polynucleotide may also be delivered by adenovirus wherein it is present within the adenovirus particle, for example, as described below.

In an alternative method, a high-efficiency nucleic acid delivery system that uses receptor-mediated endocytosis to earn DNA macro molecules into cells is employed. This is accomplished by conjugating the iron-transport protein transferrin to polycations that bind nucleic acids. Human transferrin, or the chicken homologue conalbumin, or combinations thereof is covalently linked to the small DNA-binding protein protamine or to poly lysines of various sizes through a disulfide linkage. These modified transferrin molecules maintain their ability to bind their cognate receptor and to mediate efficient iron transport into the cell. The transferrin-polycation molecules form electrophoretically stable complexes with DNA constructs or other genetic constructs of the invention independent of nucleic acid size (from short oligonucleotides to DNA of 21 kilo base pairs). When complexes of transferrin-polycation and the DNA constructs or other genetic constructs of the invention are supplied to the tumour cells, a high level of expression from the construct in the cells is expected.

High-efficiency receptor-mediated delivery of the DNA constructs or other genetic constructs of the invention using the endosome-disruption activity of defective or chemically inactivated adenovirus particles produced by the methods of Cotten et al (1992) Proc. Natl. Acad. Sci. USA 89, 6094-6098 may also be used. This approach appears to rely on the fact that adenoviruses are adapted to allow release of their DNA from an endosome without passage through the lysosome, and in the presence of, for example transferrin linked to the DNA construct or other genetic construct of the invention, the construct is taken up by the cell by the same route as the adenovirus particle.

This approach has the advantages that there is no need to use complex retroviral constructs; there is no permanent modification of the genome as occurs with retroviral infection; and the targeted expression system is coupled with a targeted delivery system, thus reducing toxicity to other cell types.

It will be appreciated that “naked DNA” and DNA complexed with cationic and neutral lipids may also be useful in introducing the DNA of the invention into cells of the individual to be treated. Non-viral approaches to gene therapy are described in Ledley (1995) Human Gene Therapy 6, 1129-1144.

Alternative targeted delivery systems are also known such as the modified adenovirus system described in WO 94/10323 wherein, typically, the DNA is carried within the adenovirus, or adenovirus-like, particle. Michael et al (1995) Gene Therapy 2, 660-668 describes modification of adenovirus to add a cell-selective moiety into a fibre protein. Mutant adenoviruses which replicate selectively in p53-deficient human tumour cells, such as those described in Bischoff et al (1996) Science 274, 373-376 are also useful for delivering the genetic construct of the invention to a cell. Thus, it will be appreciated that a further aspect of the invention provides a virus or virus-like particle comprising a genetic construct of the invention. Other suitable viruses, viral vectors or virus-like particles include lentivirus and lentiviral vectors, HSV, adeno-assisted virus (AAV) and AAV-based vectors, vaccinia and parvovirus.

A second aspect of the invention provides the use of an inhibitor of GSK-3, or polynucleotide which encodes an inhibitor of GSK-3, in the preparation of a medicament for combating prostate cancer.

The invention includes the use of an inhibitor of GSK-3 or polynucleotide which encodes an inhibitor of GSK-3, and a further anti-cancer agent, in the preparation of a medicament for combating prostate cancer.

The invention includes the use of an inhibitor of GSK-3 or polynucleotide which encodes an inhibitor of GSK-3 in the preparation of a medicament for combating prostate cancer, in an individual who is administered a further anti-cancer agent. Thus, the individual may have been administered the further anti-cancer agent previously, or is administered the further anti-cancer agent simultaneously with the medicament, or is administered further anti-cancer agent after the medicament.

The invention also includes the use of a further anti-cancer agent (other than an inhibitor of GSK-3 or polynucleotide which encodes an inhibitor of GSK-3) in the preparation of a medicament for combating prostate cancer, in an individual who is administered an inhibitor of GSK-3 or a polynucleotide which encodes an inhibitor of GSK-3. Thus, the individual may have been administered the inhibitor of GSK-3 or polynucleotide which encodes an inhibitor of GSK-3 previously, or is administered the inhibitor of GSK-3 or polynucleotide which encodes an inhibitor of GSK-3 simultaneously with the medicament, or is administered the inhibitor of GSK-3 or polynucleotide which encodes an inhibitor of GSK-3 after the medicament.

Preferences for the prostate cancer, the individual, the further anti-cancer agent, routes of administration, formulations, and so on, in this and subsequent aspects of the invention are as described above with respect to the first aspect of the invention. In an embodiment, the further anti-cancer agent is not TRAIL.

A third aspect of the invention provides a method of inhibiting prostate cancer cell proliferation in a mammalian individual, the method comprising administering an inhibitor of GSK-3, or polynucleotide which encodes an inhibitor of GSK-3, to the individual.

A fourth aspect of the invention provides the use of an inhibitor of GSK-3, or polynucleotide which encodes an inhibitor of GSK-3, in the preparation of a medicament for inhibiting prostate cancer cell proliferation.

The invention includes the use of an inhibitor of GSK-3 or polynucleotide which encodes an inhibitor of GSK-3, and a further anti-cancer agent, in the preparation of a medicament for inhibiting prostate cancer cell proliferation.

The invention includes the use of an inhibitor of GSK-3 or polynucleotide which encodes an inhibitor of GSK-3 in the preparation of a medicament for inhibiting prostate cancer cell proliferation, in an individual who is administered a further anti-cancer agent. Thus, the individual may have been administered the further anti-cancer agent previously, or is administered the further anti-cancer agent simultaneously with the medicament, or is administered further anti-cancer agent after the medicament.

The invention also includes the use of a further anti-cancer agent (other than an inhibitor of GSK-3 or polynucleotide which encodes an inhibitor of GSK-3) in the preparation of a medicament for inhibiting prostate cancer cell proliferation in an individual who is administered an inhibitor of GSK-3 or a polynucleotide which encodes an inhibitor of GSK-3. Thus, the individual may have been administered the inhibitor of GSK-3 or polynucleotide which encodes an inhibitor of GSK-3 previously, or is administered the inhibitor of GSK-3 or polynucleotide which encodes an inhibitor of GSK-3 simultaneously with the medicament, or is administered the inhibitor of GSK-3 or polynucleotide which encodes an inhibitor of GSK-3 after the medicament.

A fifth aspect of the invention provides a method of inhibiting prostate cancer cell growth ex vivo, the method comprising administering an inhibitor of GSK-3, or polynucleotide which encodes an inhibitor of GSK-3, to the prostate cancer cell.

The prostate cancer cell may be an established prostate cancer cell line or may be a primary culture from a prostate cancer biopsy.

A sixth aspect of the invention provides a composition comprising a GSK-3 inhibitor and a further anti-cancer agent. The composition may be a pharmaceutical composition. The invention thus includes a composition comprising a GSK-3 inhibitor and an anti-androgen for use in medicine. Typically, the composition is for combating prostate cancer.

Preferences for the GSK-3 inhibitor and the further anti-cancer agent are as described above with respect to the first aspect of the invention. In an embodiment, the further anti-cancer agent is not TRAIL. Preferably, the further anti-cancer agent is an anti-androgen. Preferred anti-androgens include bicalutamide and flutamide. Alternatively, the anti-cancer agent is a GnRH analogue. Preferred GnRH analogues include GnRH agonists such as leuprorelin, goserelin, and buserelin.

All of the documents referred to herein are incorporated herein, in their entirety, by reference.

The listing or discussion of a prior-published document in this specification should not necessarily be taken as an acknowledgement that the document is part of the state of the art or is common general knowledge

The invention is now described in more detail by reference to the following, non-limiting. Figures and Examples.

### BRIEF DESCRIPTION OF THE DRAWINGS

FIG. 1: Inhibition of AR transcriptional activity by Axin.

(a) Schematic illustration of the Axin constructs used. P denotes the L521 P mutation that disrupts binding of Axin to GSK-3. The numbers indicate the constructs used in (b).

(b) Requirement of the GSK-3-binding domain (but not the β-catenin- or APC-binding domains) for inhibition of AR activity by Axin. CWR-R1 prostate cancer cells were transfected with GFP (1 and 2), GFP-Axin (3), GFP-AxinP (4), GFP-Axin^{ΔAPC} (5) or GFP-Axin^{ΔAPC/Δβ-catenin} (6), MMTV-Luciferase and RSV-β-Gal. AR transcriptional activity was determined in extracts from cells grown in hormone-depleted medium in the absence (−) or presence (+) of 10 nM R1881.

(c) The GSK-3-interaction domain of Axin (GID, also known as AX2) is sufficient for inhibition of AR activity. CWR-R1 cells were transfected with empty vector (1 and 2), AX2 (3), AX2P (4) or AX2 plus pMT23 GSK-3 S9A (5), MMTV-Luciferase and RSV-β-Gal. Empty vector (pMT23) was included in transfections 1 to 4 to allow direct comparison with transfection 5. AR transcriptional activity was determined in extracts from cells grown in hormone-depleted medium either in the absence (−) or presence (+) of 10 nM R1881. All experiments were done three or more times in triplicate. The error bars indicate standard deviation.

FIG. 2: Depletion of endogenous β-catenin does not inhibit endogenous AR transcriptional activity in prostate cancer cells.

(a) HCT116 colon cancer cells, CWRR1 cells and LNCaP cells were transfected with the reporter vector pOT-Luciferase, which measures β-catenin/Tcf transcriptional activity, RSV-β-Gal, and either Control 1 (1, 3 and 5) or β-catenin (2, 4 and 6) siRNA expression vectors. β-catenin/Tcf transcriptional activity was determined in extracts from cells grown in normal growth medium. Results are presented as the activity relative to each cell line transfected with Control 1 siRNA expression vector.

(b) HCT116 cells, CWR-R1 cells and LNCaP cells were transfected with MMTV-Luciferase, RSV-β-Gal, pSG5 AR(HCT116 cells only) and either Control 1 (1, 3, 5) or β-catenin (2, 4, 6) siRNA expression vectors. AR transcriptional activity was determined in extracts from cells grown in androgen depleted medium in the presence of 10 nM (CWR-R1 cells) or 1 nM (HCT116 and LNCaP cells) R1881. Results are presented as the activity relative to each cell line transfected with Control 1. β-catenin siRNA expression vector significantly increased AR activity in CWR-R1 cells (P=0.004) and LNCaP cells (P=0.003) and significantly decreased it in HCT116 cells (P=0.01).

(c) HCT116 cells were transfected with MMTV-Luciferase, RSV-β-Gal, pSG5 AR and either Control 1 (1, 2), β-catenin (3, 4) or Control 2 (5, 6) siRNA expression vectors. AR transcriptional activity was determined in extracts from cells grown in androgen-depleted medium in the absence (−) or presence (+) of 1 nM R1881.

(d) 22Rv1 cells were transfected with MMTV-Luciferase, RSV-β-Gal and either Control 1 (1, 2), β-catenin (3, 4) or Control 2 (5, 6) siRNA expression vectors. AR transcriptional activity was determined in extracts from cells grown in androgen-depleted medium in the absence (−) or presence (+) of 1 nM R1881. All experiments were done three or more times in triplicate. The error bars indicate standard deviation.

(e) HCT116 cells were transfected with pSG5 AR and either Control 1 (lanes 1 and 2), β-catenin (lanes 3 and 4) or Control 2 (lanes 5 and 6) siRNA expression vectors and grown in androgen-depleted medium in the absence (−) or presence (+) of 1 nM R1881 for 24 h. Extracts were probed for β-catenin (upper panel) and then stripped and reprobed for AR (lower panel, upper band). The faster migrating band in the anti-AR blot is a degradation product of AR.

FIG. 3: GSK-3 increases AR transcriptional activity.

(a) 22Rv1 cells and were transfected with empty vector (1, 2), wild-type GSK-3 (3, 4), GSK-3 S9A (5, 6) or GSK-3 K216R (7, 8) plus MMTV-Luciferase, and RSV-β-Gal. AR transcriptional activity was determined in extracts from cells grown in hormone-depleted medium either in the absence (−) or presence (+) of 1 nM R1881.

(b) LNCaP cells were transfected with the indicated amounts of empty vector (1, 2), wild-type GSK-3 (3-6), GSK-3 S9A (7-10) or GSK-3 K216R (11, 12) plus MMTV-Luciferase, and RSV-â-Gal. AR transcriptional activity was determined in extracts from cells grown in hormone-depleted medium either in the absence (−) or presence (+) of 1 nM R1881. AR activity was significantly increased by wild-type GSK-3 at the higher dose (P=0.02) and by GSK-3 S9A at the lower dose (P=0.0006) and the higher dose (P=0.0004). Experiments were done twice in triplicate and error bars indicate standard deviation.

FIG. 4: Inhibition of GSK-3 reduces AR transcriptional activity.

(a) HEK293 cells were transfected with pOT-Luciferase, RSV-β-Gal and either GFP control vector (1), GFP-FRAT (2) or GFP-FRATΔC (a deletion mutant of FRAT that lacks the GSK-3-binding site) (3). β-catenin/Tcf transcriptional activity was determined in cell extracts from cells grown in normal growth medium 24 h after transfection.

(b) CWR-R1 cells were transfected with MMTV-Luciferase, RSV-β-Gal and either GFP control vector (1, 2), GFP-FRAT (3, 4) or GFP-FRATΔC (5, 6). AR activity transcriptional activity was measured in extracts from cells grown in hormone-depleted medium in the absence (−) or presence (+) of 10 nM R1881.

(c) CWR-R1 cells were transfected with pOT-Luciferase and RSV-β-Gal. Cells were treated for 24 h with carrier (1), 20 μM SB415286 (2) or 5 μM SB216763 (3) and β-catenin/Tcf transcriptional activity was determined in extracts from cells grown in normal growth medium.

(d) CWR-R1 cells were transfected with MMTV-Luciferase and RSV-β-Gal. After transfection, cells were incubated in hormone-depleted medium in the absence (−) or presence (+) of 10 nM R1881 and either carrier (1, 2), 20 μM SB415286 (3, 4) or 5 μM SB216763 (5, 6) for 24 h. AR transcriptional activity was then determined from cell extracts. All experiments were done three or more times in triplicate. The error bars indicate standard deviation.

FIG. 5: Inhibition of GSK-3 reduces prostate cancer cell growth.

(a) CWR-R1 cells were grown in the presence of carrier (ut). 5 μM SE216763 (SB21) or 20 μM SB415286 (SB41) for up to 6 days and the number of cells was counted. The experiment was done twice in triplicate and the error bars indicate standard deviation. The difference in the number of cells in untreated and treated cells was statistically significant (P=0.02 for SB216763 and P=0.008 for SB415286 at day 6).

(b) CWRR1 cells were grown for 72 h in complete growth medium in the presence of the indicated concentrations of SB216763 and the number of cells was counted. The experiment was done in triplicate and the error bars indicate standard deviation. The difference in the number of cells in untreated and treated cells was significant (P=0.0007 at 3 μM).

(c) CWR-R1, 22Rv1, DU145, PC3 and LNCaP cells were grown in normal growth medium (DUI145, PC3 and CWR-R1 cells) or in hormone-depleted medimn (22Rv1 and LNCaP cells) in the presence of 1 nM R1881 either with carrier (ut) or 5 μM SB216763 (21). The number of cells was counted after 72 h (or after 5 days for LNCaP cells). Experiments were done in triplicate and the error bars indicate standard deviation. The number of CWR-R1, 22Rv1 and LNCaP cells was significantly reduced by treatment with SB216763 (P=0.002 for LNCaP cells).

(d) 22Rv1 cells were grown in hormone-depleted medium in the absence of hormone (1 and 4), in the presence of 10⁻¹² M R1881 (2 and 5) or 10⁻⁹ M R1881 (3 and 6) and either with carrier (1-3) or 5 μM SB216763 (4-6). The number of cells was counted after 72 h. Experiments were done in triplicate and the error bars indicate standard deviation.

FIG. 6: Inhibition of GSK-3 leads to a reduction in AR protein levels. CWR-R1 cells were treated either with carrier (ut, lane 1), 5 μM SB216763 (SB21, lane 2) or with 20 μM SB415286 (SB41, lane 3) for 24 h. Western blots of whole cell extracts were probed for AR (upper panels) and reprobed for γ-tubulin as an internal loading control (lower panels).

FIG. 7: Association between AR and GSK-3 and its disruption by AX2.

(a) Extracts from COS7 cells transfected with AR and myc epitope-tagged GSK-3 were immunoprecipitated with polyclonal control antibody or polyclonal anti-AR antibody and probed with anti-myc antibody (9E10). The arrow indicates the position of GSK-3 in the cell extract.

(b) Extracts from COS 7 cells transfected with AR and myc epitope-tagged GSK-3 were immunoprecipitated with control mAb or 9E10 antibodies and probed with anti-AR antibody. The arrow indicates the position of AR in the cell extract.

(c) Extracts from COS7 cells transfected with AR and myc epitope-tagged GSK-3 and either AX2 or AX2P were immuno-precipitated with 9E10 antibodies, probed with anti-AR antibody and then reprobed with 9E10. The upper arrow indicates the position of AR and the lower arrow the position of GSK-3 in cell extracts. The band migrating above GSK-3 is IgG recognised by the secondary antibody.

### EXAMPLES

### Materials and Methods

Plasmids

GFP-Axin constructs, pOT-luciferase and RSV-β-Gal have been described (Giannini et al, 2000; Orme et al, 2003). MMTV-luciferase and pSG5 AR were gifts from Charlotte Bevan (Imperial College, London). pTER and pTERβi (van de Wetering et al, 2003) were generously provided by Marc van de Wetering and Hans Clevers (Hubrecht Lab, Utrecht, the Netherlands).

The pTER Control 1 siRNA plasmid expresses an siRNA with no known homology to human genes. It was generated using the following oligonucleotides (5′ to 3′):—

(SEQ ID NO: 3)

GATCCCCTTCTCCGAACGTGTCACGTTTCAAGAGAACGTGACACGTTC
GGAGAATTTTTGGAAA,
and

(SEQ ID NO: 4)

GGGAAGAGGCTTGCACAGTGCAAAGTTCTCTTGCACTGTGCAAGCCTC
TTAAAAACCTTTTCGA.

The pTER Control 2 siRNA plasmid expresses an siRNA to a human gene (NM_{—}004626). It was generated using the following oligonucleotides (5′ to 3′):—

(SEQ ID NO: 5)

GATCCCCGGACTCGGAACTCGTCTATTTCAAGAGAATAGACGAGTTCC
GAGTCCTTTTTGGAAA,
and

(SEQ ID NO: 6)

GGGCCTGAGCCTTGAGCAGATAAAGTTCTCTTATCTGCTCAAGGCTCA
GGAAAAACCTTTTCGA.

The annealed oligonucleotides were phosphorylated using T4 polynucleotide kinase and ligated into pTER that had been cut with BglII and HinDIII and dephosphorylated using calf intestinal phosphatase.

AX2 (FlagAx-(501-560)), AX2P, FRAT and GSK-3β constructs (Franca-Koh et al, 2002; Fraser et al, 2002; Smalley et al, 1999) were generously provided by Trevor Dale (Cardiff School of Biosciences, UK).

Cell Culture and Growth Assays

Cell lines were from the American Type Culture Collection (Rockville, Md.), except for CWR-R1 cells (Gregory et al, 2001), which were kindly provided by Christopher Gregory (University of North Carolina at Chapel Hill N.C.). Cells were grown at 37° C., 5% CO2. COS7, HEK-293 and HCT-116 cells were grown in DMEM (Invitrogen) with 10% Fetal Bovine Serum (FBS, Invitrogen) and antibiotics (100 U/ml Penicillin, 100 μg/ml Streptomycin, Sigma). LNCaP, PC3 and DU145 cells were grown in RPMI-1640 medium (Invitrogen) with 10% FBS. CWR-R1 cells were grown in Richter's Improved MEM, Zn option (Invitrogen) with 20 ng/ml EGF, 10 mM nicotinamide, 5 μg/ml insulin, 5 μg/ml transferrin, 2% FBS and antibiotics. 22Rv1 cells (Sramlcosld et al, 1999) were grown in 1:1 RPMI/DMEM with 20% FCS. For experiments using R1881, cells were grown in phenol red-free medium containing 5% (LNCaP, HCT116 and 22Rv1) or 2% (CWR-R1) charcoal-stripped serum (CSS, First Link Ltd., UK). R1881 (methyltrienolone, DuPont-NEN) was used at 1 nM and control cultures received an equal volume of carrier (ethanol). The GSK-3 inhibitors SB216763 and SB415286 were from Sigma and Biomol Research Labs Inc. (Plymouth Meeting, Pa.), respectively.

Cell growth assays were conducted according to Gregory et al, (2001). Briefly, cells (1.5×10⁵/well) were plated in 12-well plates (three wells were used for each condition) and allowed to attach overnight. Carrier or GSK-3 inhibitors were then added and, when indicated, R1881 (or carrier) was added 30 minutes later. Cells were collected by trypsinisation at the indicated times and were counted using a Coulter Counter or using a haemocytometer.

Transfections

All cells were transfected in triplicate in 6-well tissue-culture plates. Cells were incubated in serum-free Optimem-1 (Invitrogen) prior to transfection. Cells were transfected using 3.5 μl Plus reagent, 2 μl of Lipofectamine and 1 μg DNA per well according to the manufacturers instructions (Invitrogen). For transcription assays, each well of a 6-well plate was transfected with RSV promoter-driven β-Galactosidase (200 ng for prostate cancer cell lines, 20 ng for HCT116 and HEK 293 cells), 300 ng pOT-Luciferase (or pOF-Luciferase, data not shown) or 400 ng MMTV-Luciferase. When necessary, the total amount of DNA was brought to 1 μg using empty plasmid DNA. The amounts of plasmid DNA transfected per well were 200 ng of pSG5 AR (or pSG5 vector as a control), 100 ng of GFP-Axin, GFP-Axin mutants, GFP-FRAT and GFP-FRATΔC (or GFP as a control), 600 ng of AX2, AX2P (or pcDNA3 as a control), 50 ng or 500 ng of GSK-3 β constructs (or pcDNA1 vector as a control). For RNAi experiments, cells were first transfected with 1 μg pTERβi or pTER Control 1 or Control 2, and after 24 h they were transfected with reporter vectors together with 200 ng of pTER plasmids. For GSK-3 β inhibitor experiments, cells were transfected with the reporter plasmids only. In all transfections, after incubating with transfection reagents, cells were grown in their normal growth medium for 40-42 h, or in hormone-depleted medium for 18 h, after which R1881 or ethanol was added and cells were grown for a further 24 h.

Transcription Assays

Cells were rinsed in PBS and lysed using Reporter Lysis Buffer (Promega). Luciferase and β-galactosidase assays were performed using the LucLite Plus (PerkinElmer Life Sciences) and Galactolight Plus (Applied Biosystems) kits, respectively, according to manufacturer's instructions. Plates were read on a NXT TopCount Luminometer (Packard Bioscience) and values shown are Luciferase activity normalized to β-galactosidase activity.

Cell Extraction, Immunoprecipitation and Western Blotting

Cells were grown to 50-70% confluence in 100 mm dishes or 6-well plates. Lysates were obtained using the following steps: Cells were rinsed in cold TBS, lysed in modified RIP A buffer (0.5% deoxycholate, 1% Triton X-100, 20 mM Tris pH 8.0, 0.1% SDS, 100 mM NaCl, 50 mM NaF) or Nonidet P-40 buffer (1% NP-40, 20 mM Tris pH 8.0, 150 mM NaCl, 50 mM NaF) for 10 min and centrifuged for 12 min at 15,000×g. Cell extracts were then mixed with an equal volume of SDS sample buffer (Sigma Aldrich) and heated to 95° C. for 3 min. For immunoprecipitation (IP) assays in transfected COS7 cells, cell extracts were prepared using NP-40 lysis buffer, incubated with primary antibody for 1 h on ice. This was followed by 30 min incubation with 20 μl protein A/G-agarose (Cambridge Biosciences) on a rotating wheel in the cold room. After 4 washes in lysis buffer and 1 wash in TBS, the beads were resuspended in 10 μl of SDS sample buffer and heated as above. For western blotting, extracts and IPs were separated by SDS-PAGE, transferred to nitrocellulose membrane and incubated in blocking solution (3% Fraction V BSA, 1% ovalbumin in TBS-T (20 mM Tris pH 7.5, 100 mM NaCl, 0.1% Tween-20)) for 1 hour. After probing with antibodies and washing in TBS-T, antigens were visualised using chemiluminescence (ECL, Amersham Biosciences) and exposure to film. Each experiment was repeated at least three times and the results presented are representative.

Antibodies

Western Blots were probed using antibodies at 1:1000 unless stated otherwise. The following antibodies were used for western blotting: 9E10 mAb (Sigma Aldrich), P111A rabbit anti-AR (Affinity Bioreagents), anti-β-catenin mAb (Transduction Labs) and anti-γ-tubulin mAb (Sigma Aldrich). The following antibodies were used for immunoprecipitation: P110 rabbit anti-AR (Affinity Bioreagents) at 1:50, 5 μl anti-GFP polyclonal (Kypta et al, 1996) as a control, 2 μg 9E10 and 2 μg anti-GFP mAb (Roche) as a control. HRP-conjugated secondary antibodies (Jackson Laboratories) were used at 1:10000 dilution.

### Example 1

### Inhibition of AR Transcriptional Activity by Axin

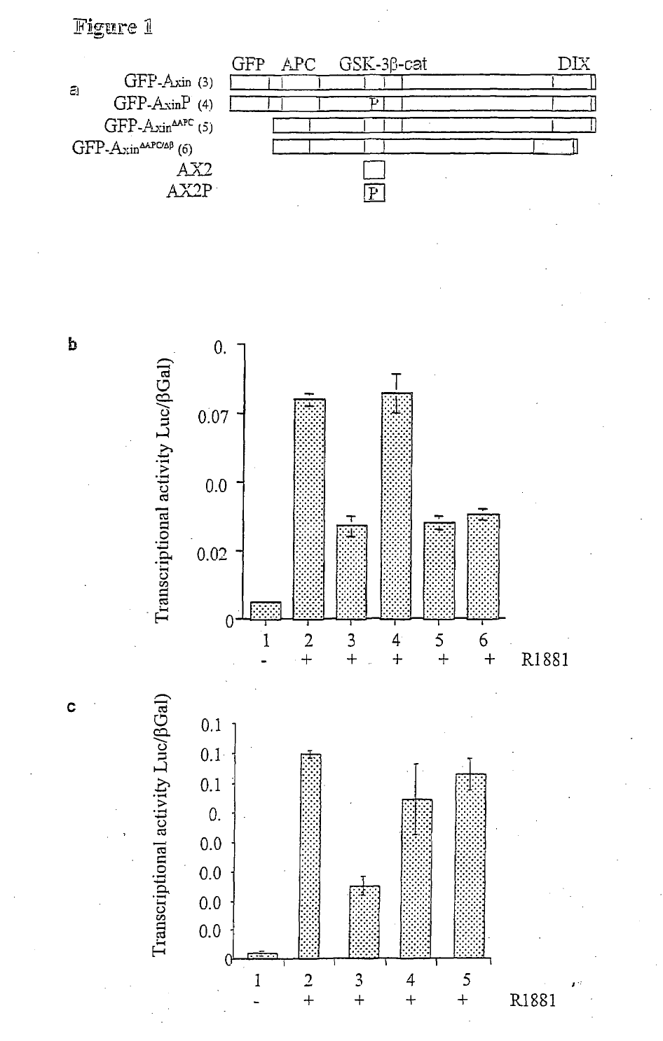

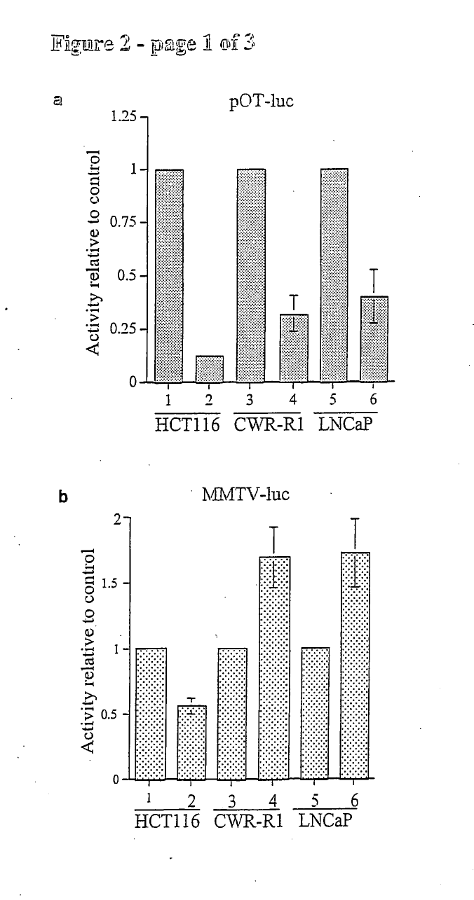

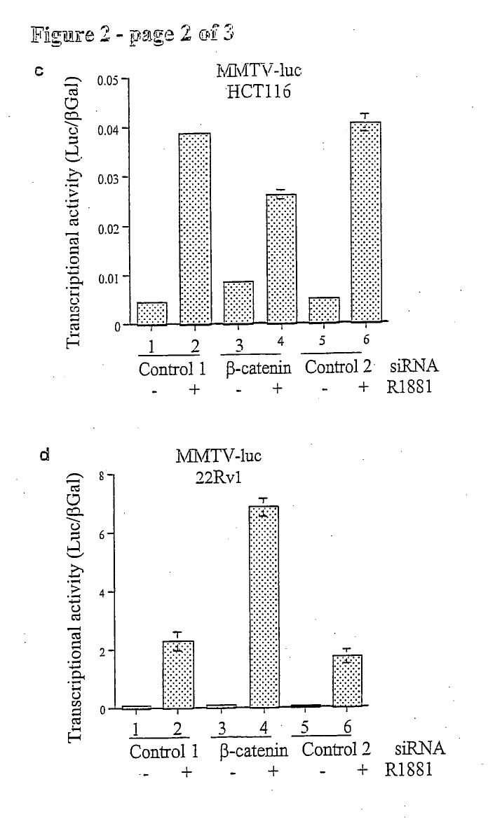

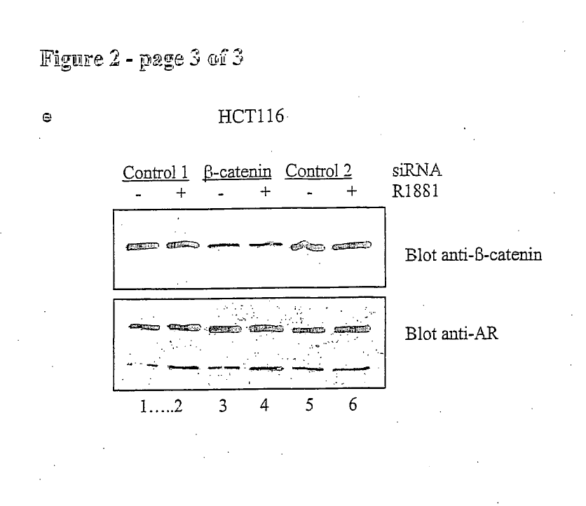

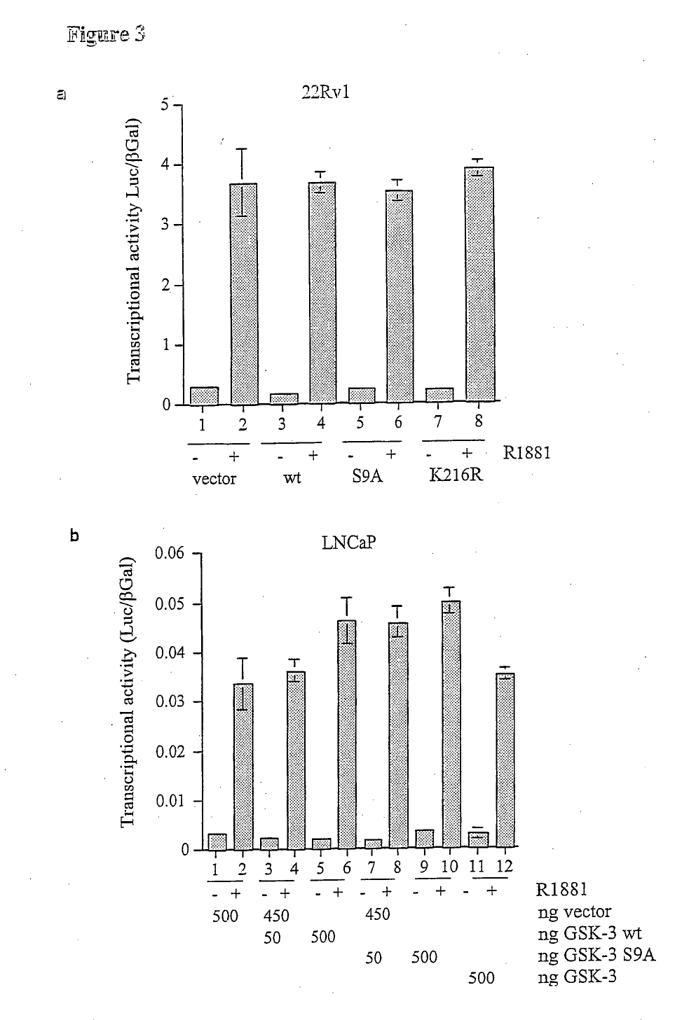

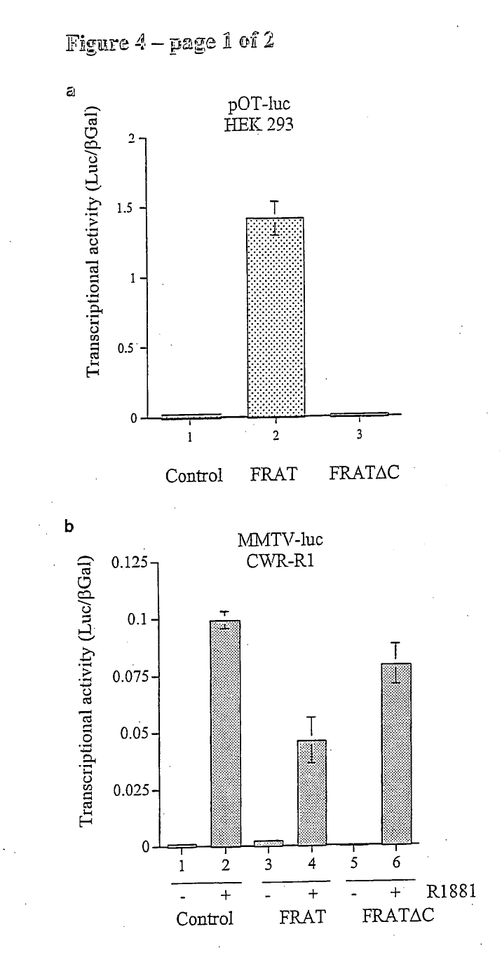

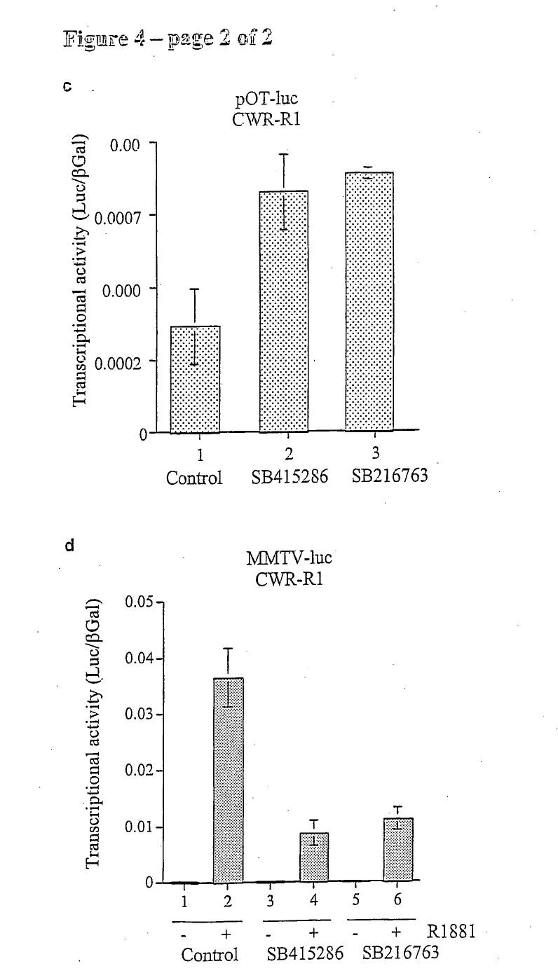

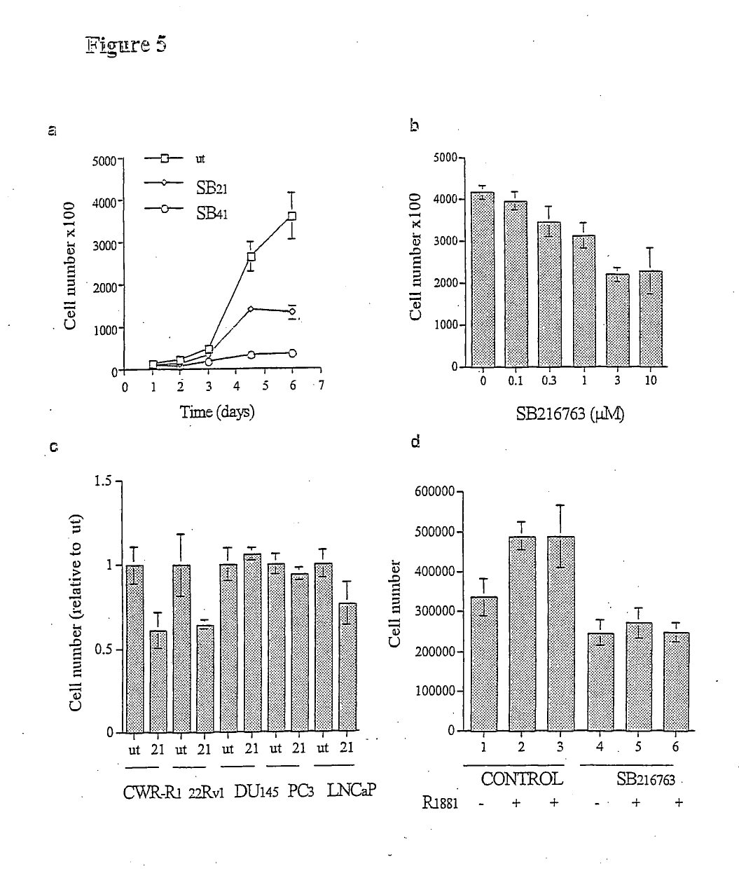

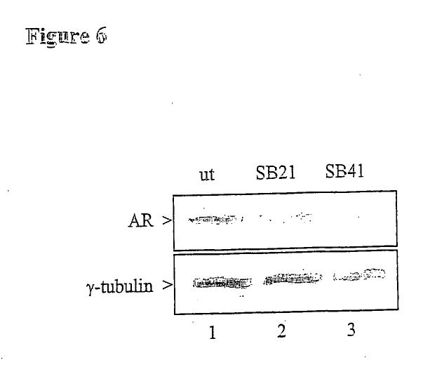

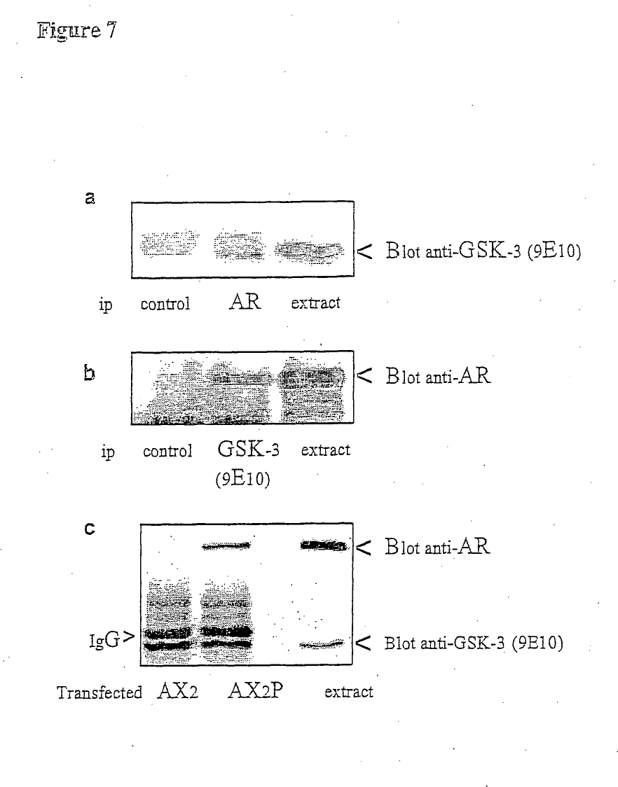

Axin inhibits the Wnt signaling pathway by acting as a scaffold protein, bringing together a number of proteins, including β-catenin, APC and GSK-3, and thereby promoting phosphorylation and degradation of β-catenin (for references see (Gregory et al, 2001; Kikuchi, 2000)). Ectopic expression of Axin is sufficient to inhibit Wnt/β-catenin signalling and is therefore often used as a tool to inhibit endogenous β-catenin function (Hsu et al., 2001; Reya et al, 2003; Ross et al, 2000). In order to determine whether endogenous β-catenin functions as a co-activator for the AR in prostate cancer cells, we expressed Axin in CWR-R1 cells. This is a cell line derived from the CWR22 xenograft model for prostate cancer that expresses endogenous AR (Gregory et al, 2001) and high levels of β-catenin (Chesire & Isaacs, 2002). For these studies we used a luciferase reporter plasmid driven by the MMTV promoter which contains androgen-receptor binding sites, R1881 (a synthetic ligand for the AR), and a panel of previously characterised GFP-Axin expression constructs (Orme et al, 2003) (FIG. 1a); GFP was used as a negative control.

As expected, compared with cells expressing GFP and treated with carrier (FIG. 1b, lane 1), addition of R1881 resulted in an increase in AR transcriptional activity (FIG. 1b, lane 2). Expression of GFP-Axin resulted in a reduction in AR transcriptional activity (FIG. 1b, lane 3). This was consistent with studies in which β-catenin overexpression has been shown to activate AR (Chesire et al, 2002; Mulholland et al, 2002; Pawlowski et al, 2002; Truica et al, 2000; Yang et al, 2002). Mutation of a conserved proline residue in the GSK-3-binding domain of Axin (GFP-AxuiP), which prevents binding to GSK-3 and also reduces binding to β-catenin (Smalley et al, 1999), prevented the inhibition of AR transcriptional activity (FIG. 1b, lane 4). To determine the importance of the β-catenin-binding domain in Axin for repression of AR, we used a mutant form of Axin that lacks both the β-catenin and the APC-binding domains, GFP-Axin^{ΔAPC/Δβ}. This mutant is useful because it cannot indirectly interact with β-catenin through endogenous APC (Hinoi et al. 2000). GFP-Axin^{ΔAPC/Δβ} inhibited AR transcriptional activity (FIG. 1b, lane 6) to the same extent as a mutant lading only the APC binding domain, GFP-Axin^{ΔAPC} (FIG. 1b, lane 5) and GFP-Axin itself.

These results suggest that the inhibition of AR transcriptional activity by Axin is independent of β-catenin and that the loss of inhibitory activity in GFP-AxinP results from its inability to bind GSK-3. In order to determine if the GSK-3-binding domain of Axin is sufficient for the inhibition of AR activity, we expressed a construct of Axin comprising only the GSK-3-binding domain, AX2 (Smalley et al, 1999). Relative to empty vector (FIG. 1c, lane 2), AX2 inhibited AR transcriptional activity (FIG. 1c, lane 3). As a control we used AX2 with a mutation in the conserved proline residue required for GSK-3 binding (AX2P), and we found that AX2P did not inhibit AR activity (FIG. 1c, lane 4). Moreover, co-expression of constitutively-active GSK-3 with AX2 rescued the inhibitory effects of AX2 on AR transcriptional activity (FIG. 1c, lane 5). Taken together, these results indicate that GSK-3, rather than β-catenin, is involved in the inhibitory effects of Axin on AR transcriptional activity.

### Example 2

### Depletion of Endogenous β-Catenin does not Inhibit Endogenous AR Transcriptional Activity in Prostate Cancer Cells

Our results using Axin suggest that endogenous β-catenin in prostate cancer cells does not affect AR activity. In order to test this possibility, we used a second approach to determine the effect of removing endogenous β-catenin on AR transcriptional activity. For these studies, we used a well-characterised β-catenin siRNA expression vector that has been shown to reduce β-catenin protein levels and inhibit β-catenin/Tcf transcriptional activity (van de Wetering et al, 2003). We first used HCT116 colon cancer cells, which have a stabilising mutation in β-catenin, and pOT-luciferase, a reporter plasmid with Tcf/LEF-1 binding sites. As expected, β-catenin siRNA inhibited β-catenin/Tcf-dependent transcription in HCT116 cells (FIG. 2a). β-catenin siRNA expression did not affect the activity of pOF-luciferase, which comprises the pOT promoter with mutations in the Tcf binding sites (data not shown). β-catenin siRNA expression also inhibited β-catenin/Tcf-dependent transcription in CWR-R1 cells, LNCaP cells and 22Rv1 cells (FIG. 2b and data not shown). Next. AR was expressed in HCT116 cells together with either control siRNA expression vector or β-catenin siRNA expression vector and MMTV-luciferase. As predicted from β-catenin overexpression studies, depletion of β-catenin resulted in a reduction in the transcriptional activity of transfected AR in HCT116 cells (FIG. 2b, lanes 1 and 2 and FIG. 2c). A second control siRNA expression vector (Control 2) had no effect on AR transcriptional activity, and the inhibitory effect was not observed in the absence of hormone (FIG. 2c). In order to determine if the effects of β-catenin siRNA resulted from a reduction in the level of expression of co-transfected AR, western blots were conducted after transfection (FIG. 2e). Expression of β-catenin siRNA led to a significant reduction in β-catenin protein (FIG. 2e, upper panel, lanes 3 and 4). The depletion of β-catenin is likely to be more efficient than suggested by the western blot since the extracts also contained β-catenin from untransfected cells, which comprise more than half the cell population. In the same extracts the expression level of AR was unaffected by expression β-catenin siRNA (FIG. 2e, lower panel), indicating that the inhibition of AR after depletion of β-catenin did not result from a reduction in AR protein levels.

We also examined the effects of depletion of β-catenin on endogenous AR activity in prostate cancer cells. The low transfection efficiency of the prostate cancer cell lines made it difficult to detect changes in β-catenin protein levels after expression of β-catenin siRNA. Therefore we used the reduction of β-catenin/Tcf transcriptional activity (FIG. 2a) as a measure of the efficiency of the β-catenin siRNA. Depletion of β-catenin did not inhibit endogenous AR transcriptional activity in CWR-R1 cells, LNCaP cells (FIG. 2b) or 22Rv1 cells (FIG. 2d); in fact AR activity was significantly increased. These results suggest that the regulation of endogenous AR transcriptional activity by endogenous β-catenin differs from what has been observed in experiments where one or both of these proteins are ectopically expressed.

### Example 3

### GSK-3 Increases AR Transcriptional Activity

Axin deletion analysis suggested an important role for GSK-3 in the regulation of AR activity. Therefore, we assessed the effects of overexpressing GSK-3 on AR transcriptional activity. For these studies we used wild-type GSK-3, a constitutively active form of GSK-3 that has a mutation at serine 9 (S9A), the inhibitory phosphorylation site, and a catalytically inactive form of GSK-3 (K216R). AR transcriptional activity was not significantly affected by expression of any of these constructs in 22Rv1 cells (FIG. 3a); GSK-3 S9A expression did result in a small increase in AR transcriptional activity in CWR-R1 cells (data not shown).

We reasoned that the weak effect of GSK-3 on AR activity might be because endogenous GSK-3 is already active in these cell lines. Therefore, we examined the effects of GSK-3 expression on AR transcriptional activity in LNCaP cells, in which GSK-3 is known to be inactive as a result of phosphorylation at serine 9 (Salas et al, 2004). When expressed at high levels, wild-type GSK-3 significantly increased AR transcriptional activity in LNCaP cells (FIG. 3b, lane 6). Constitutively active GSK-3 increased AR transcriptional activity both at low and high levels of expression (FIG. 3b, lanes 8 and 10 respectively). These results suggest that wild-type GSK-3 is inhibited by phosphorylation at serine 9 in LNCaP cells. Catalytically inactive GSK-3 did not affect AR transcriptional activity (FIG. 3b, lane 12). Taken together, these results show that GSK-3 positively regulates AR transcriptional activity in prostate cancer cells.

### Example 4

### Inhibition of GSK-3 Reduces AR Transcriptional Activity

In order to determine whether the inhibition of AR transcriptional activity by Axin was specific to the effects of Axin on GSK-3 and could not be elicited by other means, we used two further approaches. First, we expressed the proto-oncogene FRAT, which activates the Wnt signalling pathway by binding and sequestering GSK-3 (Yost et al, 1998). Consistent with the published data (Franca-Koh et al, 2002; Li et al, 1999), expression of FRAT increased β-catenin/Tcf-dependent transcription in HEK 293 cells, while expression of a FRAT mutant that cannot bind to GSK-3 (FRATΔC) did not (FIG. 4a). Expression of FRAT and FRATΔC did not affect the activity of a reporter with mutated Tcf binding sites (Smalley et al, (1999) and data not shown).

We next determined the effects of FRAT expression on AR transcriptional activity in CWR-R1 cells. As predicted from the experiments using Axin, FRAT inhibited AR transcriptional activity, and the extent of inhibition was significantly reduced when using FRATΔC (FIG. 4b). However, FRATΔC did repress AR activity to a certain extent, particularly when expressed at higher levels (data not shown). We interpret this result as a manifestation of an indirect effect on GSK-3, since FRATΔC can associate with disheveled, which binds to GSK-3 via Axin (Li et al, 1999). To summarise, although FRAT and Axin have opposite effects on β-catenin/Tcf transcriptional activity, they both inhibit AR transcriptional activity, and in both cases this requires their GSK-3 binding domains.

In a second approach, we used two commercially available inhibitors of GSK-3, SB415286 and SB216763 (Coghlan et al, 2000). First we examined the effects of these inhibitors on β-catenin/Tcf-dependent signalling. CWR-R1 cells were transfected with the reporter vector pOT-Luc and treated with GSK-3 inhibitors for 24 h. Consistent with results in other cell types (Coghlan et al, 2000), both inhibitors increased β-catenin/Tcf-dependent transcriptional activity (FIG. 4c). In contrast, both inhibitors reduced AR transcriptional activity (FIG. 4d), consistent with a model in which endogenous GSK-3 activates AR. Taken together, these results show that the inhibitory effects of Axin on AR result from its ability to regulate GSK-3, rather than any function unique to Axin.

### Example 5

### Inhibition of GSK-3 Reduces Prostate Cancer Cell Growth

We next examined the effects of GSK-3 inhibitors on prostate cancer cell growth. We first used CWR-R1 cells, which are hypersensitive to androgens and grow optimally in medium containing 2% FCS, as described previously (Gregory et al, 2001). CWR-R1 cells were treated with GSK-3 inhibitors and counted over a period of six days (FIG. 5a). Both SB415286 and SB216763 repressed CWR-R1 cell growth. The inhibitory effects of SB216763 on cell growth were maximal at 3 μM (FIG. 5b), which is the same concentration that is optimal for the ability of this drug to protect neurons from apoptotic cell death (Cross et al, 2001). The inhibitory effects of SB415286 increased with dose up to the maximal dose tested (50 μM; data not shown).

In order to determine whether inhibition of GSK-3 specifically inhibited growth of All-positive prostate cancer cells, we compared the effects of SB216763 on the growth of CWR-R1, 22Rv1 and LNCaP cells, which express AR, and DU145 and PC3 cells, which do not (FIG. 5c). 22Rv1 and CWR-R1 cells both derive from the CWR22 prostate cancer xenograft but were selected under different growth conditions (Gregory et al, 2001; Sramkoski et al, 1999). SB216763 similarly reduced the growth of CWR-R1 cells and 22Rv1 cells. In contrast, SB216763 did not significantly affect DU145 or PC3 cell growth, consistent with the possibility that AR is required for the growth inhibitory response. The growth of LNCaP cells was weakly inhibited by SB216763, consistent with the low GSK-3 activity in this cell line. Taken together, these results show that inhibition of GSK-3 reduces the growth of AR-positive prostate cancer cells.

To determine if inhibition of GSK-3 specifically affected androgen-dependent cell growth, a similar experiment was conducted using 22Rv1 cells grown in hormone-depleted medium in the absence or presence of R1881 (FIG. 5d). Although 22Rv1 cells are able to grow in hormone-depleted medium, their growth can be stimulated by androgens (Sramkoski et al, 1999). We found that R1881 stimulated the growth of 22Rv1 cells and that this was blocked by treatment with SB216763. However, SB216763 also inhibited hormone-independent proliferation of 22Rv1 cells to a certain extent.

### Example 6

### Inhibition of GSK-3 Leads to a Reduction in AR Protein Levels

As a first step in determining the mechanism by which GSK-3 regulates AR transcriptional activity, we examined the expression level of the AR protein in CWRR1 cells treated with GSK-3 inhibitors. CWR-R1 cells were treated with GSK-3 inhibitors for 24 h and whole cell extracts were probed for AR by western blotting (FIG. 6, upper panel). Interestingly, compared with untreated cells (lane 1), the protein level of AR was reduced after treatment with both SB216763 (lane 2) and SB415286 (lane 3). SB415286 appeared to reduce AR protein levels more than SB216763, but reprobing the blot for tubulin (lower panel) indicated that part of this reduction resulted from the effects of this drug on the number of cells. Nevertheless, taking into account the loading controls, both inhibitors reduced AR protein levels in CWR-R1 cells.

### Example 7

### Association Between AR and GSK-3 and its Disruption by AX2

In order to determine whether the effects of GSK-3 on AR might involve interactions between these proteins, we examined the possibility that GSK-3 and AR form a complex. AR and myc epitope-tagged GSK-3 were co-expressed in COS7 cells and these proteins were then immunoprecipitated and probed by western blotting (FIG. 7). GSK-3 was detected in anti-AR immune precipitates and not in control immune precipitates (FIG. 7a), and AR was detected in anti-GSK-3 immune precipitates and not in control immune precipitates (FIG. 7b). These results support a model in which GSK-3 increases AR transcriptional activity by forming a complex with AR. To determine a possible mechanism for the inhibition of AR activity by Axin, we expressed AX2 or AX2P in COS7 cells together with GSK-3 and AR (FIG. 7c). AR was readily detected in GSK-3 immune precipitates from COS7 cells expressing AX2P. In contrast, we were unable to detect a complex between AR and GSK-3 in cells expressing AX2. This suggests that AX2 inhibits AR transcriptional activity by preventing interaction between GSK-3 and AR and further supports a model in which the association of GSK-3 with AR leads to elevated AR transcriptional activity.

### Discussion of Examples 1-7

Several reports have suggested that β-catenin is a transcriptional co-activator of AR (Chesire et al, 2002; Mulholland et al, 2002; Truica et al, 2000; Yang et al, 2002). However, the results from our experiments using Axin and a β-catenin siRNA expression vector to deplete β-catenin suggest that endogenous β-catenin, although highly expressed in prostate cancer cell lines, is not a transcriptional co-activator for endogenous AR. The siRNA experiments were performed in HCT116 cells using wild-type AR, suggesting that the different responses of ectopic and endogenous AR to depletion of β-catenin might result from mutations in endogenous AH in LNCaP and CWR-R1 cells. However, we obtained similar results in HCT116 cells using both wild-type and the LNCaP mutant form of AR (unpublished observations). Our observations highlight the importance of confirming results obtained using ectopically expressed proteins by examining the functions of the endogenous proteins. Clearly, further experiments are necessary to determine the function of endogenous β-catenin in the regulation of AR transcriptional activity.

Our results using Axin suggest a role for GSK-3 in the regulation of AR transcriptional activity. The repression of AR activity by Axin required an intact GSK-3 binding domain, since a point mutation in this domain prevented repression. Indeed, expression of the GSK-3 binding domain alone was sufficient to repress AR activity, and the repression of AR by this domain was rescued by co-expression of constitutively active GSK-3. Furthermore, the GSK-3-binding domain of Axin prevented the formation of a GSK-3-AR complex. In support of the hypothesis that Axin inhibits AR transcriptional activity by binding to GSK-3, a second GSK-3-binding protein, FRAT, also repressed AR activity. Further work will be required to determine if endogenous Axin or FRAT play a role in the regulation of AR transcriptional activity, or if the observations we have made solely result from their ability to associate with GSK-3 when they are overexpressed.

The role of GSK-3 in the regulation of AR transcriptional activity and prostate cancer cell growth was addressed further using GSK-3 inhibitors. The results of these experiments confirmed that GSK-3 activity was required for maximal AR transcriptional activity and proliferation in CWR-R1 cells and 22Rv1 cells. Interestingly, treatment of CWR-R1 cells with GSK-3 inhibitors reduced the level of AR protein. One possible interpretation of these data is that GSK-3 directly phosphorylates AR, and that this phosphorylation increases AR stability. GSK-3 has been shown to regulate the stability of a number of proteins (for references see (Doble & Woodgett, 2003; Frame & Cohen, 2001; Woodgett, 2001)). In the majority of cases, phosphorylation by GSK-3 promotes degradation of its target substrate (examples include β-catenin, cyclin D1 and c-myc). However, there are also examples where phosphorylation by GSK-3 promotes protein stability, such as Axin (Yamarnoto et al, 1999). Finally, GSK-3 can have both positive and negative effects on protein stability; for example, it stabilizes Nuclear Factor-κB1/p105 under resting conditions and primes p105 for degradation upon TNF-a treatment (Demarchi et al, 2003). To continue this line of reasoning, the decrease in AR transcriptional activity in cells treated with GSK-3 inhibitors would result from a reduction in AR protein levels, as was observed experimentally (FIG. 6). Although GSK-3 inhibits the transcriptional activity of many nuclear proteins (for references see (Doble & Woodgett, 2003; Frame & Cohen, 2001; Woodgett, 2001)), it activates at least one transcription factor, CREB, by direct phosphorylation (Salas et al, 2003). It remains to be seen whether GSK-3 activates the AR by direct phosphorylation, and whether this then leads to increased AR protein levels. However, our observation that AR and GSK-3 can be coimmunoprecipitated supports such a possibility.

Three other groups have recently reported results from experiments examining the regulation of AR by GSK-3. Salas et al (2004) showed that GSK-3 phosphorylates AR and inhibits AR transcriptional activity in transfected COS-1 cells. Wang et al (2004) reported similar results using both COS-1 cells and LNCaP cells, and they also showed that the inhibition of AR by GSK-3 is blocked by lithium chloride. In contrast, Liao et al (2004) showed that inhibition of GSK-3 using either chemical inhibitors or GSK-3 siRNA reduces AR transcriptional activity. Interestingly, this group also found that depletion of β-catenin by siRNA increased AR transcriptional activity. Our results, obtained using different chemical inhibitors of GSK-3 and using the GSK-3-binding proteins Axin and FRAT, are in agreement with the report from Liao et al. It is possible that the use of different systems for transcription assays (endogenous AR in prostate cancer cell lines as opposed to transfected AR in COS-1 cells) accounts for many of the differences between our results and those suggesting that GSK-3 inhibits AR.

The results of Wang et al (2004) in LNCaP cells also differ from ours; it is possible that they reflect differences in cell passage number, since this is known to influences regulation of AR activity by Akt, a kinase that can inhibit GSK-3 (Lin et al 2003). Akt inhibits AR transcriptional activity in low passage LNCaP cells but enhances AR activity in high passage LNCaP cells. Our experiments were restricted to low passage LNCaP cells (below 25 passages), consistent with the inhibitory effects of Akt on AR activity. Another important observation made by Wang et al is that expression of an inducible form of GSK-3 S9A in CWR22 cells (which originate from the same patient as CWR-R1 and 22Rv1 cells) inhibits their growth. This is in contrast to our observations that inhibition of GSK-3 reduces CWR-R1 and 22Rv1 cell growth. The difference might reflect the different methods used to determine cell number (cell counting versus MTT assay).

To summarise, we have used protein and chemical inhibitors of GSK-3 to show that GSK-3 activity is required for maximal activity of the AR, and that inhibition of GSK-3 leads to a reduction both in AR protein levels and the growth of certain prostate cancer cell lines. This suggests a novel therapeutic application for GSK-3 inhibitors in the treatment of prostate cancer.

### Example 8

### Treatment of a Patient with Prostate Cancer by Administering an Inhibitor of GSK-3

A patient with prostate cancer is treated with intravenous infusions of saline solutions of a pharmaceutical composition comprising a GSK-3 inhibitor. The infusions are administered weekly for a time of 3 to 6 months.

### Example 9

### Treatment of a Patient with AD Prostate Cancer by Administering an Inhibitor of GSK-3 and an Anti-Androgen

A patient with AD prostate cancer is treated with intravenous infusions of saline solutions of a pharmaceutical composition comprising a GSK-3 inhibitor and an anti-androgen. The infusions are administered weekly for a-time of 3 to 6 months.

### REFERENCES

1. Assikis & Simons (2004) Semin Oncol 31 (2 Suppl. 4): 26-32.
2. Bhat, R. V., Stanley, J., Correll, M. P., Fieles, W. E., Keith, R. A., Scott, C. W. & Lee, C. M. (2000). Proc Natl Acad Sci USA, 97, 11074-9.
3. Cheshire, D. R. & Isaacs, W. B. (2003). Endocr Relat Cancer, 10, 537-60.
4. Chesire. D. R. Ewing, C. M., Gage, W. R. & Isaacs, W. B. (2002). Oncogene, 21.2679-94.
5. Chesire, D. R. & Isaacs, W. B. (2002). Oncogene, 21, 8453-69.
6. Cline et al (2002) Diabetes 51:2903-2910
7. Coghlan, M. P., Culbert, A. A., Cross, D. A. et al (2000). Chem Biol, 1, 793-803.
8. Cronauer, M. V., Schulz, W. A., Burchardt, T., Anastasiadis, A. G., de la Taille, A., Ackermann, R. & Burchardt, M. (2003). Int J Oncol, 23, 1095-102.
9. Cross, D. A., Alessi, D. R., Cohen, P., Andjelkovich, M. & Hemmings, B. A. (1995). Nature, 378, 785-9.
10. Cross, D. A., Culbert, A. A., Chalmers, K. A., Facci, L., Skaper, S. D. & Reith, A. D. (2001). J Neurochem, 77, 94-102.
11. Demarchi, F., Bertoli, C, Sandy, P. & Schneider, C. (2003). J Biol Chem, 278, 39583-90.
12. Doble, B. W. & Woodgett, J. R. (2003). J Cell Sci, 116, 1175-86.
13. Frame, S. & Cohen, P. (2001). Biochem J, 359, 1-16.
14. Franca-Koh, J., Yeo, M., Fraser, E., Young, N. & Dale, T. C. (2002). J Biol Chem, 277, 43844-8.
15. Fraser, E., Young, N., Dajani, R., Franca-Koh, J., Ryves, J., Wilhams, R. S., Yeo, M., Webster, M. T., Richardson, C, Smalley, M. J., Pearl, L. H., Harwood, A. & Dale, T. C. (2002). J Biol Chem, 277, 2176-85.
16. Fujimuro, M., Wu, F. Y., ApRhys, C., Kajumbula, H., Young, D. B., Hayward, G. S. & Hayward, S. D. (2003). Nat Med, 9, 300-6.
17. Giannini, A. L., Vivanco, M. & Kypta, R. M. (2000). J Biol Chem, 275, 21883-8,
18. Gregory, C. W., Johnson, R. T., Jr., Mohler, J. L., French, F. S. & Wilson, E. M. (2001). Cancer Res, 61, 2892-8.
19. Grimes, C. A. & Jope, R. S. (2001). Prog Neurobiol, 65, 391-426.
20. Gulley & Dahut (2004) Am J Ther 11(4): 288-94.
21. Flinoi, T., Yamamoto, H., Kishida, M., Takada, S., Kisliida, S. & Kikuchi, A. (2000). J Biol Chem, 275, 34399-406.
22. Hosbi. M., Taliashima, A., Noguchi K., Mufayama. M., Sato. M., Kondo. S., Saitoh, Y., Ishiguro, K., Hoshino, T. & Imahori. K. (1996). Proc Natl Acad Sci USA, 93, 2719-23 .
23. Hsu, W., Shakya, R. & Costantini F. (2001). J Cell Biol 155, 1055-64.
24. Ikeda, S., Kishida, S., Yamamoto, H., Murai, H., Koyama, S. & Kikuchi, A. (1998). Embo J, 17, 1371-84.
25. Kikuchi A. (2000). Biochem Biophys Res Commun, 268, 243-8.
26. Kypta, R. M., Su, H. & Reichardt, L. F. (1996). J Cell Biol 134, 1519-29.
27. Li L., Yuan, H., Weaver, C. D., Mao, J., Farr, G. H., 3rd, Sussman, D. J., Jonkers, J., Kimelman, D. & Wu, D. (1999). Embo J, 18, 4233-40.
28. Liao, X, Thrasher, J. B., Holzbeierlein, L, Stanley, S. & Li, B. (2004). Endocrinology, 145(6): 2941-9.
29. Lin, H. K., Hu, Y. C., Yang, L., Altuwaijri, S., Chen, Y. T., Kang, H. Y. & Chang, C. (2003). J Biol Chem, 278, 50902-7.
30. Mulholland, D. J., Cheng, H., Reid, K, Rennie, P. S. & Nelson, C. C (2002). J Biol Chem, 277, 17933-43 .
31. Orme, M. H., Giannini, A. L., Vivanco, M. D. & Kypta, R. M. (2003). Mol Cell Neurosci, 24, 673-86.
32. Pawlowski IE., Ertel, J. R., Allen, M. P., Xu, M., Butler, C, Wilson, E. M. & Wierman, M. E. (2002). J Biol Chem, 277, 20702-10.
33. Reya, T., Duncan, A. W., Allies, L., Domen, J., Scherer, D. C., Willert, K., Hintz, L., Nusse, R. & Weissman, I. L. (2003). Nature, 423, 409-14.
34. Ring et al (2003) Diabetes 52:588-595
35. Ross, S. E., Hernati, N., Longo, K. A., Bennett, C. N., Lucas, P. C., Erickson, R. L. & MacDougald, O. A. (2000). Science, 289, 950-3.
36. Salas, T. R., Kirn, J., Vakar-Lopez, F., Sabichi, A. L., Troncoso, P., Jenster, G., Kilcuclri, A, Chen, S. Y., Shemshedini L., Suraokar, M., Logothetis, C. J., DiGiovanni, J., Lippman, S. M. & Menter, D. G. (2004). J Biol Chem, 279(18): 19191-200.
37. Salas, T. R., Reddy, S. A., Clifford, J. L., Davis, R. J., Kikuchi, A., Lippman, S. M. & Menter, D. G. (2003). J Biol Chem, 278, 41338-46.
38. Schellhammer & Davis (2004) Clin Prostate Cancer 2(4): 213-9.
39. Smalley, M. J., Sara. E., Paterson, H., Naylor. S., Cook, D. Jayatilake, H., Fryer, L. G., Hutchinson, L., Fry, M. J. & Dale, T. C. (1999). Embo J, 18, 2823-35.
40. Sramkoski, R. M., Pretlow, T. G., 2nd, Giaconia, J. M., Pretlow, T. P., Schwartz, S., Sy, M. S., Marengo, S. R., Rhim, J. S., Zhang, D. & Jacobberger, J. W. (1999). In Vitro Cell Dev Biol Anim, 35, 403-9.
41. Stambolic, V. & Woodgett, J. R. (1994). Biochem J, 303 (Pt 3), 701-4.
42. Truica, C. I., Byers, S. & Gelmann, E. P. (2000). Cancer Res, 60, 4709-13 .
43. van de Wetering, M., Oving, I., Muncan, V., Pon Fong, M. T., Brantjes, H., van Leenen, D., Holstege, F. C., Bmmmelkamp, T. R., Agarni, R. & Clevers, H. (2003). EMBO Rep, 4, 609-15.
44. Wang, L., Lin, H. K., Hu, Y. C., Xie, S., Yang, L. & Chang, C. (2004). J Biol Chem, 279(31): 32444-52.
45. Wilding, G., Gelmann, E. P. & Freter, C. E. (1990). Prostate, 16, 15-27.
46. Woodgett, J. R. (2001). Sci STKE, 2001, RE12.
47. Yamamoto, F. I., Kishida, S., Kishida, M., Ikeda, S., Takada, S. & Kikuchi, A. (1999). J Biol Chem, 274, 10681-4.
48. Yang, F., Li, X., Sharma, M., Sasaki, C. Y., Longo, D. L., Lim, B. & Sun, Z. (2002). J Biol Chem, 277, 11336-44.
49. Yost, C, Farr, G. H., 3 rd, Pierce, S. B., Ferkey, D. M., Chen, M. M. & Kimelman, D. (1998). Cell, 93, 1031-41 .
50. Barry et al (2003) FEBS Lett. 553:173.
51. Bhat, R, et al (2003) J. Biol. Chem. 278, 45937.
52. Knockaert et al (2004) Oncogene 23: 4400.
53. Kunick et al (2004) Bioorg. Med. Chem. Lett. 14: 413.
54. Martinez et al (2002) J. Med. Chem. 45: 1292
55. Meijer et al (2003) Chem. Biol 10: 1255.
56. Naerum et al (2002) Bioorg. Med. Chem. Lett. 12: 1525.
57. O'Neill, et al (2004) Bioorg. Med. Chem. 12: 3167.
58. Pierce et al (2005) J. Med. Chem. 48(4): 1278-81.
59. Plotkin et al (2003) J. Pharmacol Exp. Ther. 305: 974.
60. Polychronopoulos et al (2004) J. Med. Chem. 47: 935.
61. Sato et al (2004) Nat. Med. 10: 55.

1. A method of combating prostate cancer in a mammalian individual the method comprising administering an inhibitor of glycogen synthase kinase-3 (GSK-3), or a polynucleotide which encodes an inhibitor of GSK-3, to the individual.

2. (canceled)

3. A method of inhibiting prostate cancer cell proliferation in a mammalian individual, the method comprising administering an inhibitor of GSK-3, or a polynucleotide which encodes an inhibitor of GSK-3, to the individual.

4. (canceled)

5. (canceled)

6. The method according to claim 1 comprising a prior step of determining whether the prostate cancer expresses AR.

7. The method or a use according to claim 1 wherein the prostate cancer expresses the androgen receptor (AR).

8. The method or a use according to claim 1 wherein the prostate cancer is androgen dependent.

9. The method or a use according to claim 1 wherein the prostate cancer is androgen independent.

10. The method or a use according to claim 1 wherein the inhibitor of GSK-3 is a selective GSK-3 inhibitor.

11. The method according to claim 1 wherein the inhibitor of GSK-3 is selected from the group consisting of SB415286; a related GSK-3 inhibitory compound; a 3-indolyl-4-phenyl-1H-pyrrole-2,5-dione derivative; SB216763; GSK-3-binding domain of Axin or a variant thereof that inhibits GSK-3; GSK-3-binding domain of FRAT or a variant thereof that inhibits GSK-3; CHIR 98023; CHIR 99021; CHIR 99014; RO318220; GF10923X; and a GSK-3 specific siRNA molecule.

12. The method according to claim 1 further comprising administering an additional anti-cancer agent.

13. (canceled)

14. The method according to claim 12 wherein the additional anti-cancer agent is an anti-androgen, or a GnRH agonist.

15. The method according to claim 12 wherein the additional anti-cancer agent is a steroid or a chemotherapy agent.

16. The method or a use according to claim 12 wherein the additional anti cancer agent is different from TRAIL.

17. A composition comprising a GSK-3 inhibitor and an anti-androgen or a GnRH analogue.

18. (canceled)

19. (canceled)

20. (canceled)

21. The composition according to claim 17 wherein the anti-androgen is bicalutamide or flutamide.

22. A The composition according to claim 17 wherein the GnRH analogue is a GnRH agonist.

23. The composition according to claim 17 wherein the GSK-3 inhibitor is a selective GSK-3 inhibitor.

24. The method according to claim 3 comprising the a prior step of determining whether the prostate cancer cell expresses AR.

25. The method according to claim 3 wherein the prostate cancer cell expresses the androgen receptor (AR).

26. The method according to claim 3 wherein the prostate cancer cell is androgen dependent.

27. The method according to claim 3 wherein the prostate cancer cell is androgen independent.

28. The method according to claim 3 wherein the inhibitor of GSK-3 is a selective GSK-3 inhibitor.

29. The method according to claim 3 wherein the inhibitor of GSK-3 is selected from the group consisting of SB415286; a related GSK-3 inhibitory compound; a 3-indolyl-4-phenyl-1H-pyrrole-2,5-dione derivative; SB216763; GSK-3-binding domain of Axin or a variant thereof that inhibits GSK-3; GSK-3-binding domain of FRAT or a variant thereof that inhibits GSK-3; CHIR 98023; CHIR 99021; CHIR 99014; RO318220; GF10923X; and a GSK-3 specific siRNA molecule.

30. The method according to claim 3 further comprising administering an additional anti-cancer agent.

31. The method according to claim 30 wherein the additional anti-cancer agent is an anti-androgen or a GnRH agonist.

32. The method according to claim 30 wherein the additional anti-cancer agent is a steroid or a chemotherapy agent.

33. The method according to claim 30 wherein the additional anti cancer agent is different from TRAIL.

34. The method according to claim 14 wherein the anti-androgen comprises bicalutamide or flutamide, and the GnRH agonist comprises leuprovelin, guserelin, or buserelin.

35. The method according to claim 15 wherein the steroid comprises hydrocortisone, prednisone, or dexamethasone, and the chemotherapy agent comprises mitozantrone, estramustine, or docetaxol.

36. The composition according to claim 22 wherein the GnRH analogue comprises leuproreun, goserelin and buserelin.


SEQUENCE LISTING (6 sequences)

SEQ ID NO: 1 - DNA, 21 bp, Artificial Sequence
  GSK-3b inhibitor
aagaatcgagagctccagatc

SEQ ID NO: 2 - DNA, 21 bp, Artificial Sequence
  GSK-3b inhibitor
aagtaatccacctctggctac

SEQ ID NO: 3 - DNA, 64 bp, Artificial Sequence
  oligonucleotide used to generate control 1 siRNA
gatccccttctccgaacgtgtcacgtttcaagagaacgtgacacgttcggagaatttttggaaa

SEQ ID NO: 4 - DNA, 64 bp, Artificial Sequence
  oligonucleotide used to generate control 1 siRNA
gggaagaggcttgcacagtgcaaagttctcttgcactgtgcaagcctcttaaaaaccttttcga

SEQ ID NO: 5 - DNA, 64 bp, Artificial Sequence
  oligonucleotide used to generate control 2 siRNA
gatccccggactcggaactcgtctatttcaagagaatagacgagttccgagtcctttttggaaa

SEQ ID NO: 6 - DNA, 64 bp, Artificial Sequence
  oligonucleotide used to generate control 2 siRNA
gggcctgagccttgagcagataaagttctcttatctgctcaaggctcaggaaaaaccttttcga